SEQUENCE LISTING (1 sequence)

SEQ ID NO: 1 - DNA, 156 bp, Homo sapiens
  source 1..156: mol_type = genomic DNA
tataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacata

(19) United States
(12) Patent Application Publication
(10) Pub. No.: US 2026/0125728 A1
(43) Pub. Date: May 7, 2026

(54) DNA PRODUCTION METHODS

(71) Applicant: FLAGSHIP PIONEERING INNOVATIONS VII, LLC, Cambridge, MA (US)

(72) Inventors: Jenny Patricia England, Arlington, MA (US); Pedro Rivera-Pomales, Woburn, MA (US)

(21) Appl. No.: 19/381,568

(22) Filed: Nov. 6, 2025

Related U.S. Application Data
(60) Provisional application No. 63/717,426, filed on Nov. 7, 2024.

(51) Int. Cl. C12P 19/34

(52) U.S. Cl. CPC C12P 19/34

(57) ABSTRACT

The disclosure provides, for example, a method of making a composition comprising purified DNA molecules. In some embodiments, the method comprises contacting a solution comprising a plurality of circular, double-stranded DNA molecules to a hydrophobic interaction chromatography matrix. In some embodiments, the circular, double-stranded DNA molecules lack one or both of a bacterial origin of replication and a selectable marker.

### RELATED APPLICATIONS

This application claims priority to U.S. Ser. No. 63/717,426, filed Nov. 7, 2024, the entire contents of which are incorporated herein by reference.

### SEQUENCE LISTING

The instant application contains a Sequence Listing which has been submitted electronically in XML format and is hereby incorporated by reference in its entirety. Said XML copy, created on Oct. 29, 2025, is named F2128-702010_SL.xml and is 1,850 bytes in size.

### BACKGROUND

There is a need for novel therapeutic modalities, and methods of manufacturing, to address unmet medical need.

### SUMMARY OF THE INVENTION

Described herein are pharmaceutical DNA compositions, constructs, preparations, methods of using such compositions, constructs and preparations, and methods of making the same.

### Enumerated Embodiments

1. A method of making a composition comprising purified DNA molecules, the method comprising:
   a) providing a solution comprising a plurality of circular, double-stranded DNA (cdsDNA) molecules, wherein the cdsDNA molecules lack one or both of a bacterial origin of replication and a selectable marker;
   b) contacting the solution to a hydrophobic interaction chromatography (HIC) matrix; and
   c) collecting at least some of the cdsDNA molecules from the matrix; thereby making the composition comprising purified DNA molecules.
2. A method of making a composition comprising purified DNA molecules, the method comprising:
   a) providing a solution comprising a plurality of circular, double-stranded DNA (cdsDNA) molecules;
   b) subjecting the solution to conditions having exonuclease activity;
   c) contacting the solution to a hydrophobic interaction chromatography (HIC) matrix; and
   d) collecting at least some of the cdsDNA molecules from the matrix, thereby making the composition comprising purified DNA molecules.
3. A composition (e.g., a pharmaceutical composition) comprising circular, double-stranded DNA (cdsDNA) molecules, wherein:
   i) the cdsDNA molecules lack one or both of a bacterial origin of replication and a selectable marker; and
   ii) at least 85%, at least 90%, at least 95%, at least 96%, at least 97%, at least 98%, at least 99%, or at least 99.5% by mass of total DNA molecules in the composition are the cdsDNA molecules.
4. The method of embodiment 1 or 2, or the composition of embodiment 3, wherein the cdsDNA molecules are not supercoiled.
5. The method of any of embodiments 1, 2, or 4, or the composition of embodiment 3 or 4, wherein the cdsDNA molecules substantially lack a material portion of a vector backbone (e.g., plasmid backbone).
6. The method of any of embodiments 1, 2, 4, or 5, or the composition of any of embodiments 3-5, wherein at least 85%, at least 90%, at least 95%, at least 96%, at least 97%, at least 98%, at least 99%, at least 99.5%, or at least 99.6% by mass of total DNA molecules in the composition are the cdsDNA molecules.
7. The method any of embodiments 1, 2, or 4-6, further comprising measuring one or more (e.g., two, three, four, five, six, or all) of the following in the composition comprising purified DNA molecules:
   (a) the mass % of cdsDNA molecules;
   (b) the mass % of linear DNA molecules;
   (c) the mass % of linear concatemeric DNA molecules;
   (d) the mass % of circular concatemeric DNA molecules;
   (e) the length, e.g., average length, of cdsDNA molecules;
   (f) endotoxin levels; or
   (g) amount of protein, e.g., enzyme (e.g., nuclease) levels or activity.
8. The method of embodiment 7, further comprising formulating the composition as a drug product if the one or more measured parameters (a)-(g) meet a predetermined value (e.g., a quality release specification).
9. The method of any of embodiments 1, 2, or 4-8, or the composition of any of embodiments 3-6, wherein 85%-90%, 90%-95%, 95%-96%, 96%-97%, 97%-98%, 98%-99%, or 99%-99.6% by mass of total DNA molecules in the composition are the cdsDNA molecules.
10. The method of any of embodiments 1, 2, or 4-9, or the composition of any of embodiments 3-6 or 9, wherein the composition comprises less than:
   15%, 10%, 5%, 4%, 3%, 2%, 1%, or 0.5% of linear DNA molecules by mass;
   15%, 10%, 5%, 4%, 3%, 2%, 1%, or 0.5% of linear concatemeric DNA molecules by mass; and/or
   15%, 10%, 5%, 4%, 3%, 2%, 1%, or 0.5% of circular concatemeric DNA molecules by mass.
11. The method of any of embodiments 1, 2, or 4-10, or the composition of any of embodiments 3-6, 9, or 10, wherein the composition comprises:
   0.5%-1%, 1%-2%, 2%-3%, 3%-4%, 5%-10%, or 10%-15% of linear DNA molecules by mass;
   0.5%-1%, 1%-2%, 2%-3%, 3%-4%, 5%-10%, or 10%-15% of linear concatemeric DNA molecules by mass; and/or
   0.5%-1%, 1%-2%, 2%-3%, 3%-4%, 5%-10%, or 10%-15% of circular concatemeric DNA molecules by mass.
12. The method of any of embodiments 1, 2, or 4-11, or the composition of any of embodiments 3-6 or 9-11, wherein the composition is substantially free of (e.g., free of) endotoxin.
13. The method of any of embodiments 1, 2, or 4-12, or the composition of any of embodiments 3-6 or 9-12, wherein the composition is substantially free of (e.g., free of) protein.
14. The method of any of embodiments 1 or 4-13, which comprises a step of subjecting the solution to conditions having exonuclease activity.
15. The method of any of embodiments 2 or 4-14, wherein the step of subjecting the solution to conditions having exonuclease activity is performed prior to the step of contacting the solution to the HIC matrix.
16. The method of any of embodiments 2 or 4-15, wherein the step of subjecting the solution to conditions having exonuclease activity comprises contacting (e.g., incubating) the solution with an exonuclease.
17. The method of embodiment 16, wherein the exonuclease is a double-stranded DNA exonuclease (e.g., T5 exonuclease).
18. The method of any of embodiments 2 or 4-17, wherein the step of subjecting the solution to conditions having exonuclease activity is performed with 60-100 rpm, e.g., 70-90 rpm, e.g., about 80 rpm, agitation.
19. The method of any of embodiments 2 or 4-18, wherein the step of subjecting the solution to conditions having exonuclease activity is performed in a buffer comprising 100-300 mM Tris, e.g., at pH of 8-12, e.g., at pH of about 10.
20. The method of any of embodiments 2 or 4-19, wherein the step of subjecting the solution to conditions having exonuclease activity is performed in a buffer comprising 1-5 mM Tris, e.g., about 2 mM Tris, e.g., at pH of 8-12, e.g., at pH of about 10.
21. The method of any of embodiments 2 or 4-20, wherein the step of subjecting the solution to conditions having exonuclease activity is performed in a buffer comprising 30-70 mM (e.g., about 50 mM) potassium acetate, 10-30 mM (e.g., about 20 mM) Tris-acetate, 5-15 mM (e.g., about 10 mM) magnesium acetate, and 0.5-2 mM (e.g., about 1 mM DTT), and wherein the buffer has a pH of 7-8 (e.g., about 7.9) at about 25° C.
22. The method of any of embodiments 1, 2, or 4-21, wherein the solution of a) further comprises a second cdsDNA molecule that comprises a bacterial origin of replication and a selectable marker.
23. The method of any of embodiments 1, 2, or 4-22, wherein prior to contacting the solution to the HIC matrix, the solution comprises 20-50 mM Tris, 1.0-2.0 M ammonium sulfate, and 5-15 mM EDTA, and wherein the solution has a pH of 7.0-8.0.
24. The method of any of embodiments 1, 2, or 4-23, wherein prior to contacting the solution to the HIC matrix, the solution comprises 20-30 mM Tris, 1.0-1.5 M ammonium sulfate, and 7.5-12.5 mM EDTA, and wherein the solution has a pH of 6.5-7.5.
25. The method of any of embodiments 1, 2, or 4-24, wherein prior to contacting the solution to the HIC matrix, the solution comprises about 25 mM Tris, about 1.25 M ammonium sulfate, and about 10 mM EDTA, and wherein the solution has a pH of about 7.0.
26. The method of any of embodiments 1, 2, or 4-25, which further comprises, prior to contacting the solution to the HIC matrix, a step of contacting the HIC matrix with an equilibration buffer comprising 20-50 mM Tris, 1.0-2.0 M ammonium sulfate, and 5-15 mM EDTA, and wherein the equilibration buffer comprises a pH of 7.0-8.0.
27. The method of any of embodiments 1, 2, or 4-26, which further comprises, prior to contacting the solution to the HIC matrix, a step of contacting the HIC matrix with an equilibration buffer comprising 20-30 mM Tris, 1.0-1.5 M ammonium sulfate, and 7.5-12.5 mM EDTA, and wherein the equilibration buffer comprises a pH of 6.5-7.5.
28. The method of any of embodiments 1, 2, or 4-27, which further comprises, prior to contacting the solution to the HIC matrix, a step of contacting the HIC matrix with an equilibration buffer comprising about 25 mM Tris, about 1.25 M ammonium sulfate, and about 10 mM EDTA, and wherein the equilibration buffer comprises a pH of about 7.0.
29. The method of any of embodiments 26-28, wherein the HIC matrix is contacted with 5-20, e.g., 8-12, e.g., about 10, column volumes (CV) of the equilibration buffer, optionally at a 2-10 (e.g., about 5) mL/CV flow rate.
30. The method of any of embodiments 1, 2, or 4-29, wherein prior to contacting the solution to the HIC matrix, the solution is contacted with a filter, e.g., a 0.1-0.5 m filter, e.g., a 0.2-0.3 μm filter, e.g., a 0.22 m filter.
31. The method of any of embodiments 1, 2, or 4-30, wherein collecting at least some of the cdsDNA molecules from the matrix comprises collecting a flow-through from the matrix, wherein the flow-through comprises the cdsDNA molecules.
32. The method of any of embodiments 1, 2, or 4-31, which further comprises, following collecting at least some of the cdsDNA molecules from the matrix, a step of filtering and/or concentrating the cdsDNA molecules.
33. The method of embodiment 32, wherein filtering the cdsDNA molecules comprises contacting the cdsDNA molecules with a 0.22-0.45 m filter.
34. The method of embodiment 32 or 33, wherein concentrating the cdsDNA molecules comprises contacting the cdsDNA molecules with a 10-50 (e.g., 10-30) kDa molecular weight cut-off (MWCO) hollow fiber, e.g., with a Repligen system.
35. The method of any of embodiments 1, 2, or 4-34, wherein, following collecting at least some of the cdsDNA molecules from the matrix, the cdsDNA molecules are contacted with a solution comprising sodium citrate.
36. The method of embodiment 35, wherein the solution comprising sodium citrate comprises 0.5 mM to 2 mM sodium citrate (e.g., 1 mM sodium citrate) and has a pH between 5.5 and 7.0 (e.g., between 6.0 and 6.5 or between 6.0 and 7.0, e.g., about 6.5).
37. The method of any of embodiments 1, 2, or 4-36, wherein the solution of a) comprises: less than about 20%, 15%, or 10% of cdsDNA molecules by mass;
   at least about 55%, 65%, or 75% of linear DNA molecules by mass;
   at least about 15%, 18%, or 20% of linear concatemeric DNA molecules by mass; and/or
   at least about 1%, 2%, or 3% of circular concatemeric DNA molecules.
38. The method of any of embodiments 1, 2, or 4-37, wherein the HIC matrix comprises a butyl (C4) ligand.
39. The method of any of embodiments 1, 2, or 4-38, wherein the HIC matrix comprises poly(butyl methacrylate-co-ethylene dimethacrylate).
40. The method of any of embodiments 1, 2, or 4-39, wherein the HIC matrix is comprised in a column (e.g., a monolithic column).
41. The method of any of embodiments 1, 2, or 4-40, wherein the mass of cdsDNA molecules in the composition comprising purified DNA molecules is at least 50%, at least 60%, at least 65%, at least 70%, at least 74%, at least 75%, at least 80%, at least 85%, at least 90%, at least 95%, at least 98%, at least 99%, or at least 99.4% of the mass of cdsDNA molecules in the solution prior to contacting the solution to the HIC matrix.
42. The method of any of embodiments 1, 2, or 4-41, wherein the mass of cdsDNA molecules in the composition comprising purified DNA molecules is 50%-60%, 60%-65%, 65%-70%, 70%-75%, 75%-80%, 80%-85%, 85%-90%, 90%-95%, 95%-99%, or 99%-99.5% of the mass of cdsDNA molecules in the solution prior to contacting the solution to the HIC matrix.
43. The method of any of embodiments 1, 2, or 4-42, or the composition of any of embodiments 3-6 or 9-13, wherein the composition comprises a A260/280 ratio of 1.8-2.02, e.g., 1.8-1.9 or 1.9-2.02, e.g., about 1.89, about 1.91, about 1.92, about 1.99, about 2.01, or about 2.02.
44. The method of any of embodiments 1, 2, or 4-43, or the composition of any of embodiments 3-6, 9-13, or 43, wherein the composition comprises a A260/230 ratio of 2.0-2.3 or 2.0-2.4, e.g., 2.0-2.1, 2.1-2.3, or 2.3-2.4, e.g., about 2.02, about 2.14, about 2.29, about 2.30, about 2.36, or about 2.32.
45. The method of any of embodiments 1, 2, or 4-44, or composition of any of embodiments 3-6, 9-13, 43, or 44, wherein the composition comprises between 1 g and 500 g, between 500 μg and 1 mg, between 1 mg and 5 mg, between 5 mg and 10 mg, between 10 mg and 15 mg, or between 10 mg and 20 mg of the cdsDNA molecules.
46. The method of any of embodiments 1, 2, or 4-45, which does not comprise a step of contacting the solution with a restriction enzyme.
47. The method of any of embodiments 1, 2, or 4-46, which does not comprise a step of subjecting the solution to thermal denaturation conditions.
48. The method of any of embodiments 1, 2, or 4-47, which does not comprise a step of performing anion exchange chromatography.
49. The method of any of embodiments 1, 2, or 4-48, which does not comprise a step of performing affinity chromatography.
50. The method of any of embodiments 1, 2, or 4-49, wherein the solution of a) is not from a cell lysate.
51. The method of any of embodiments 1, 2, or 4-50, wherein the solution of a) was not produced in a microorganism.
52. The method of any of embodiments 1, 2, or 4-51, wherein the solution of a) was produced in a cell-free system.
53. The method of any of embodiments 1, 2, or 4-52, wherein the solution of a) was produced by a polymerase chain reaction (PCR).
54. The method of any of embodiments 1, 2, or 4-53, wherein the solution of a) is substantially free of host cell proteins.
55. The method of any of embodiments 1, 2, or 4-54, or the composition of any of embodiments 3-6, 9-13, or 43-45, wherein the cdsDNA molecules have a length of at least 500, at least 1000, at least 2000, at least 3000, at least 4000, at least 5000, at least 6000, at least 7000, at least 8000, at least 9000, or at least 10000 nucleotides.
56. The method of any of embodiments 1, 2, or 4-55, or the composition of any of embodiments 3-6, 9-13, 43-45, or 55, wherein the cdsDNA molecules have a length of between 500-1000, 1000-2000, 2000-3000, 3000-4000, 4000-5000, 5000-6000, 6000-7000, 7000-8000, 8000-9000, or 9000-10000 nucleotides.
57. The method of any of embodiments 1, 2, or 4-56, or the composition of any of embodiments 3-6, 9-13, 43-45, 55, or 56, wherein the cdsDNA molecules comprise a promoter sequence and an effector sequence that encodes an effector (e.g., a therapeutic effector).
58. The method or composition of embodiment 57, wherein the effector is a polypeptide (e.g., a DNA binding protein, an epigenetic modifying factor, an antigen, a hormone, an enzyme, a CRISPR-linked enzyme, a mobile genetic element protein, a gene writer, an antibody, a signaling peptide, a receptor ligand, a receptor (e.g., a chimeric antigen receptor (CAR) or a T cell receptor), or a clotting factor).
59. The method or composition of embodiment 57, wherein the effector comprises an RNA (e.g., an mRNA, a tRNA, lncRNA, miRNA, rRNA, snRNA, siRNA, piRNA, snoRNA, snRNA, exRNA, scaRNA, Y RNA, or hnRNA).
60. The method of any of embodiments 1, 2, or 4-59, or the composition of any of embodiments 3-6, 9-13, 43-45, or 55-59, wherein one, two, or all of:
   i) the effector sequence does not encode a viral protein;
   ii) the cdsDNA molecules do not comprise a viral packaging signal; or
   iii) the cdsDNA molecules do not comprise a viral ITR.
61. The method of any of embodiments 1, 2, or 4-60, or the composition of any of embodiments 3-6, 9-13, 43-45, or 55-60, wherein the cdsDNA molecules comprise a chemically modified nucleotide.
62. The method of any of embodiments 1, 2, or 4-61, or the composition of any of embodiments 3-6, 9-13, 43-45, or 55-61, wherein the cdsDNA molecules comprise a sense strand and an antisense strand.
63. The method or composition of embodiment 62, wherein the sense strand comprises the chemically modified nucleotide, and the antisense strand is substantially free of (e.g., is free of) chemically modified nucleotides.
64. The method or composition of embodiment 62 or 63, wherein 1-25%, 25-50%, 50-75%, or 75-100% of nucleotides in the sense strand are chemically modified nucleotides.
65. The method or composition of embodiment 62, wherein the antisense strand comprises the chemically modified nucleotide, and the sense strand is substantially free of (e.g., is free of) chemically modified nucleotides, optionally wherein 1-25%, 25-50%, 50-75%, or 75-100% of nucleotides in the antisense strand are chemically modified nucleotides.
66. The method or composition of embodiment 61 wherein the chemically modified nucleotide comprises a chemically modified nucleobase.
67. The method or composition of any of embodiments 62-64 or 66, wherein the sense strand comprises a chemically modified nucleobase, and the antisense strand is substantially free of (e.g., is free of) chemically modified nucleobases.
68. The method or composition of any of embodiments 62-64, 66, or 67, wherein 1-25%, 25-50%, 50-75%, or 75-100% of nucleobases in the sense strand are chemically modified nucleobases.
69. The method or composition of any of embodiments 62, 65, or 66, wherein the antisense strand comprises a chemically modified nucleobase, and the sense strand is substantially free of (e.g., is free of) chemically modified nucleobases.
70. The method or composition of embodiment 62, 65, 66, or 69, wherein 1-25%, 25-50%, 50-75%, or 75-100% of nucleobases in the antisense strand are chemically modified nucleobases.
71. The method or composition of any of embodiments 66-70, wherein the chemically modified nucleobase comprises a chemically modified cytosine nucleobase.
72. The method or composition of any of embodiments 66-70, wherein the chemically modified nucleobase comprises a canonical uracil nucleobase.
73. The method or composition of any of embodiments 66-70, wherein the chemically modified nucleobase comprises a chemically modified uracil nucleobase.
74. The method or composition of embodiment 73, wherein the chemically modified uracil nucleobase comprises 5-hydroxymethyluracil.
75. The method or composition of embodiment 61, wherein the chemically modified nucleotide comprises a chemically modified sugar.
76. The method or composition of embodiment 61, wherein the chemically modified nucleotide comprises a backbone modification (e.g., phosphorothioate).
77. The method of any embodiments 1, 2, or 4-76, or the composition of any of embodiments 3-6, 9-13, 43-45, or 55-76, wherein the cdsDNA molecules lack a bacterial origin of replication.
78. The method of any embodiments 1, 2, or 4-77, or the composition of any of embodiments 3-6, 9-13, 43-45, or 55-77, wherein the cdsDNA molecules lack a selectable marker.
79. The method of any embodiments 1, 2, or 4-78, or composition of any of embodiments 3-6, 9-13, 43-45, or 55-78, wherein the cdsDNA molecules lack both of a bacterial origin of replication and a selectable marker.
80. A composition (e.g., a pharmaceutical composition) comprising the purified DNA molecules produced by the method of any of embodiments 1, 2, or 4-79.
81. The method of any of embodiments 1, 2, or 4-79, or the composition of any of embodiments 3-6, 9-13, 43-45, or 55-80, wherein at least 85%, at least 90%, at least 95%, at least 96%, at least 97%, at least 98%, at least 99%, or at least 99.5% of the DNA molecules in the composition have substantially the same length in nucleotides.
82. The method of any of embodiments 1, 2, 4-79, or 81, or the composition of any of embodiments 3-6, 9-13, 43-45, or 55-81, wherein at least 85%, at least 90%, at least 95%, at least 96%, at least 97%, at least 98%, at least 99%, or at least 99.5% of the DNA molecules in the composition have the same length in nucleotides.
83. The method of any of embodiments 1, 2, 4-79, 81, or 82, or the composition of any of embodiments 3-6, 9-13, 43-45, or 55-82, wherein the effector sequence of all DNA molecules in the composition have substantially the same length in nucleotides (e.g., the effector sequence of all DNA molecules in the composition have the same length in nucleotides).
84. The method of any of embodiments 1, 2, 4-79, or 81-83, or the composition of any of embodiments 3-6, 9-13, 43-45, or 55-83, wherein all DNA molecules in the composition have a length of between 100, 200, 500, or 1000 nucleotides of each other.
85. The method of any of embodiments 1, 2, 4-79, or 81-84, or the composition of any of embodiments 3-6, 9-13, 43-45, or 55-84, wherein all DNA molecules in the composition have a length of between 500-1000, 1000-2000, 2000-3000, 3000-4000, 4000-5000, 5000-6000, 6000-7000, 7000-8000, 8000-9000, or 9000-10000 nucleotides.
86. The method of any of embodiments 1, 2, 4-79, or 81-85, or the composition of any of embodiments 3-6, 9-13, 43-45, or 55-85, wherein all DNA molecules in the composition encode substantially the same effector (e.g., all DNA molecules in the composition encode the same effector).
87. The method of any of embodiments 1, 2, 4-79, or 81-86, or the composition of any of embodiments 3-6, 9-13, 43-45, or 55-86, wherein all DNA molecules in the composition have substantially the same sequence (e.g., all DNA molecules in the composition have the same sequence).
88. The method of any of embodiments 1, 2, 4-79, or 81-87, which further comprises formulating the purified DNA molecules to produce a lipid nanoparticle (LNP) comprising the purified DNA molecules.
89. The composition of any of embodiments 3-6, 9-13, 43-45, or 55-87, wherein the DNA molecules, e.g., purified DNA molecules, are comprised in an LNP.
90. The composition of any of embodiments 3-6, 9-13, 43-45, or 55-87, which is substantially free of (e.g., is free of) LNPs.
91. The composition of any of embodiments 3-6, 9-13, 43-45, 55-87, or 90, which is substantially free of (e.g., free of) nanoparticles.
92. The composition of any of embodiments 3-6, 9-13, 43-45, 55-87, 90, or 91, which is substantially free of (e.g., is free) of lipids.
93. The composition of any of embodiments 3-6, 9-13, 43-45, 55-87, or 89-92, further comprising an electroporation buffer.
94. The composition of any of embodiments 3-6, 9-13, 43-45, 55-87, or 89-93, further comprising a transfection reagent.
95. The method of any of embodiments 1, 2, 4-79, or 81-88, or the composition of any of embodiments 3-6, 9-13, 43-45, 55-87, or 89-94, wherein the composition is substantially free of viral proteins.
96. The method of any of embodiments 1, 2, 4-79, 81-88, or 95, or the composition of any of embodiments 3-6, 9-13, 43-45, 55-87, or 89-95, wherein the composition is substantially free of microorganisms.
97. The method of any of embodiments 1, 2, 4-79, 81-88, 95, or 96, or the composition of any of embodiments 3-6, 9-13, 43-45, 55-87, or 89-96, wherein the composition is substantially free of bacterial proteins.
98. The method of any of embodiments 1, 2, 4-79, 81-88, or 95-97, or the composition of any of embodiments 3-6, 9-13, 43-45, 55-87, or 89-97, wherein the composition is substantially free of bacterial DNA.
99. A method of expressing an effector in a target cell, the method comprising:
   introducing into a target cell the composition of any of embodiments 3-6, 9-13, 43-45, 55-87, or 89-98, or a composition comprising the purified DNA molecules produced by the method of any of embodiments 1, 2, 4-79, 81-88, or 95-98, wherein the cdsDNA molecules comprise an effector sequence encoding an effector; and
   maintaining (e.g., incubating) the cell under conditions suitable for expressing the effector;
   thereby expressing the effector in the target cell.
100. A method of delivering an effector to a target cell, the method comprising:
   introducing into a target cell the composition of any of embodiments 3-6, 9-13, 43-45, 55-87, or 89-98, or a composition comprising the purified DNA molecules produced by the method of any of embodiments 1, 2, 4-79, 81-88, or 95-98, wherein the cdsDNA molecules comprise an effector sequence encoding an effector;
   thereby delivering the effector to the target cell.
101. A method of modulating (e.g., increasing or decreasing) a biological activity in a target cell, the method comprising:
   introducing into a target cell the composition of any of embodiments 3-6, 9-13, 43-45, 55-87, or 89-98, or a composition comprising the purified DNA molecules produced by the method of any of embodiments 1, 2, 4-79, 81-88, or 95-98, wherein the cdsDNA molecules comprise an effector sequence encoding an effector; and
   maintaining (e.g., incubating) the cell under conditions suitable for expressing the effector;
   thereby modulating the biological activity in the target cell.
102. The method of embodiment 101, wherein the effector increases the biological activity in the target cell.
103. The method of embodiment 101, wherein the effector decreases the biological activity in the target cell.
104. The method of any of embodiments 101-103, wherein the biological activity comprises cell growth, cell metabolism, cell signaling, cell movement, specialization, interactions, division, transport, homeostasis, osmosis, or diffusion.
105. The method of any of embodiments 99-104, wherein the cell is an animal cell, e.g., a mammalian cell, e.g., a human cell.
106. A method of treating a cell, tissue, or subject in need thereof, the method comprising:
   administering to the cell, tissue, or subject the composition of any of embodiments 3-6, 9-13, 43-45, 55-87, or 89-98, or a composition comprising the purified DNA molecules produced by the method of any of embodiments 1, 2, 4-79, 81-88, or 95-98, wherein the cdsDNA molecules comprise an effector sequence encoding an effector;
   thereby treating the cell, tissue, or subject.

### Definitions

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments, e.g., Fab, Fab′ and F(ab′)₂, Fd, Fvs, single-chain Fvs (scFv), rIgG, single-chain antibodies, disulfide-linked Fvs (sdFv), nanobody, fragments including either a VL or VH domain, fragments produced by an Fab expression library, and anti-idiotypic (anti-Id) antibodies. Antibodies described herein can be of any type (e.g., IgG, IgE, IgM, IgD, IgA, and IgY), class (e.g., IgG1, IgG2, IgG3, IgG4, IgAQ1 and IgA2) or subclass of immunoglobulin molecule. Moreover, unless otherwise indicated, the term “monoclonal antibody” (mAb) is meant to include both intact molecules as well as antibody fragments (such as, for example, Fab and F(ab′)2 fragments) that are capable of specifically binding to a target protein. Fab and F(ab′)2 fragments lack the Fc fragment of an intact antibody.

As used herein, the term “bacterial origin of replication” refers to a DNA sequence that can be used to initiate DNA replication in a bacterium. In some embodiments, the bacterial origin of replication has a wild-type sequence of an origin of replication in a bacterial genome.

As used herein, the term “carrier” means a compound, composition, reagent, or molecule that facilitates or promotes the transport or delivery of a composition (e.g., a DNA molecule described herein) into a cell. For example, a carrier may be a partially or completely encapsulating agent.

As used herein, the term “chemically modified nucleotide,” as used herein with respect to DNA molecules, refers to a nucleotide comprising one or more structural differences relative to the canonical deoxyribonucleotides (i.e., G, T, C, and A). A chemically modified nucleotide may have (relative to a canonical nucleotide) a chemically modified nucleobase, a chemically modified sugar, a chemically modified phosphodiester linkage, or a combination thereof. No particular process of making is implied; for instance, a chemically modified nucleotide can be produced directly by chemical synthesis, or by covalently modifying a canonical nucleotide.

As used herein, the term “chemically modified nucleobase” as used herein with respect to DNA molecules, refers to a nucleobase comprising one or more structural differences relative to the canonical nucleobases (i.e., guanine, thymine, cytosine, and adenine). No particular process of making is implied; for instance, a chemically modified nucleobase can be produced directly by chemical synthesis, or by covalently modifying a canonical nucleobase. A canonical uracil present in a DNA molecule is considered a chemically modified nucleobase under this definition.

As used herein, the term “chemically modified cytosine nucleobase,” as used herein with respect to DNA molecules, refers to a chemically modified nucleobase wherein the nucleobase comprises a monocyclic 6-member ring in which carbon 4 is covalently bound to a nitrogen that is not one of the six members of the ring, wherein the nucleobase comprises one or more structural differences relative to canonical cytosine nucleobase. In some embodiments, the C-5 position of the nucleobase can have a substitution other than H. For example, the C-5 position of the nucleobase can have a substitution of —OH; -aldehyde; -carboxylic acid; -alkyl; —(CH₂)ₘOR₃, m=1-3 and R₃═H or a sugar molecule; or -propargylamino. No particular process of making is implied.

As used herein, the term “chemically modified uracil nucleobase” as used herein with respect to DNA molecules, refers to a chemically modified nucleobase wherein the nucleobase comprises a monocyclic 6-member ring in which carbon 4 is covalently bound to an oxygen through a double bond, and wherein the nucleobase comprises one or more structural differences relative to canonical uracil and thymine nucleobases. In some embodiments, the C-5 position of the nucleobase can have a substitution other than H or a methyl group. For example, the C-5 position of the nucleobase can have a substitution of —CH₂OH; -aminoallyl; -propargylamino; or -dihydroxypentyl. No particular process of making is implied.

As used herein, the term “uracil nucleobase” encompasses both canonical uracil nucleobases and chemically modified uracil nucleobases.

As used herein the term “circular” in reference to a dsDNA molecule described herein, means a dsDNA molecule that lacks a free end. A circular dsDNA molecule may be covalently closed. The term circular does not imply that the DNA would appear as a perfect geometric circle under a microscope; for instance, a circular dsDNA molecule may be supercoiled. In some embodiments, the circular dsDNA molecule comprises a first strand that is circular and lacks a free end, and a second strand that is circular and lacks a free end, and the first strand and second strand hybridize with each other.

As used herein, the term “free end” in reference to a DNA molecule described herein, refers to an end of a DNA strand where the terminal nucleotide is covalently bound to exactly one other nucleotide.

As used herein, the term “closed end” refers to a portion of a DNA molecule positioned at one end of a double-stranded region, in which all nucleotides within the portion of the DNA molecule are covalently attached to adjacent nucleotides on either side. A closed end may, in some embodiments, include a loop comprising one or more nucleotides that are not hybridized to another nucleotide. In some embodiments, every nucleotide of the closed end is hybridized to another nucleotide. In some embodiments, a double-stranded DNA molecule comprises a first closed end (e.g., upstream of a heterologous object sequence) and a second closed end (e.g., downstream of a heterologous object sequence).

As used herein, “concatemeric DNA” of a reference DNA molecule refers to a DNA molecule that comprises a plurality of copies of the reference DNA molecule covalently linked in series, wherein each copy has at least 50% of the length of the reference DNA molecule and has a sequence identity of at least 90% to the relevant portion of the reference DNA molecule. In some embodiments, the concatemeric DNA further comprises one or more additional, shorter copies that are less than 50% of the length of the reference DNA molecule. In some embodiments, the concatemeric DNA further comprises additional DNA that is not a copy of the reference DNA molecule. In some embodiments, the concatemeric DNA is linear double-stranded DNA or circular double-stranded DNA.

As used herein, the term “open end” refers to a portion of a DNA molecule positioned at one end of a double-stranded region, in which at least one nucleotide (a “terminal nucleotide”) is covalently attached to exactly one other nucleotide. In some embodiments, the terminal nucleotide comprises a free 5′ phosphate. In some embodiments, the terminal nucleotide comprises a free 3′ OH. In some embodiments, in a double-stranded DNA molecule comprising a first DNA strand and a second DNA strand, the open end comprises a first terminal nucleotide on the first DNA strand and a second terminal nucleotide on the second DNA strand. In some embodiments, a DNA molecule comprises a first open end (e.g., upstream of a heterologous object sequence) and a second open end (e.g., downstream of a heterologous object sequence). In some embodiments, the open end comprises a blunt end, a sticky end, or a Y-adaptor.

As used herein, the term “DNA molecule” refers to any compound and/or substance that comprises at least two (e.g., at least 10, at least 20, at least 50, at least 100) covalently linked deoxyribonucleotides. In some embodiments, the DNA molecule is a single oligonucleotide chain, while in other embodiments, the DNA molecule comprises a plurality of oligonucleotide chains, while in yet other embodiments the DNA molecule is a portion of an oligonucleotide chain. In some embodiments, the DNA molecule is a compound and/or substance that is or can be incorporated into an oligonucleotide chain via a phosphodiester linkage. In some embodiments, the DNA molecule comprises solely canonical nucleotides. In some embodiments, the DNA molecule comprises one or more chemically modified nucleotides. In some embodiments, at least 80%, at least 85%, at least 90%, at least 95%, at least 98%, at least 99%, or 100% of the sugars of the DNA molecule are deoxyribose sugars. In some embodiments, the DNA molecule was prepared by one or more of: isolation from a natural source, enzymatic synthesis by polymerization based on a complementary template (in vivo or in vitro), reproduction in a recombinant cell or system, and chemical synthesis.

As used herein, the term “double-stranded DNA exonuclease” refers to an enzyme having exonuclease activity for double-stranded DNA. In some embodiments, the enzyme also has exonuclease activity for single-stranded DNA. In some embodiments, the enzyme also has endonuclease activity.

As used herein, the term “effector sequence” refers to the part of a DNA molecule that exerts a function on a cell, either directly (wherein the effector sequence is a functional DNA sequence) or by encoding a functional RNA or protein. The encoded functional RNA or protein is referred to as the “effector”.

As used herein, the term “heterologous”, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

As used herein, the term “heterologous functional sequence” refers to a nucleic acid sequence that is heterologous to an adjacent (e.g., directly adjacent) nucleic acid sequence and has one or more biological functions.

As used herein, the term “hydrophobic interaction chromatography (HIC) matrix” refers to a solid support suitable for performing HIC. In some embodiments, the matrix is a resin. In some embodiments, the matrix is comprised by a column. In some embodiments, the matrix is comprised by a monolithic column.

As used herein, the terms “increasing” and “decreasing” refer to modulating resulting in, respectively, greater or lesser amounts, of function, expression, or activity of a metric relative to a reference. For example, subsequent to administration of a DNA molecule in a method described herein, the amount of the metric described herein (e.g., the level of gene expression, or a marker of innate immunity) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration, or relative to administration of a control DNA molecule. Generally, the metric is measured subsequent to administration at a time that the administration has had the recited effect, e.g., at least one day, one week, one month, 3 months, or 6 months, after a treatment regimen has begun.

As used herein the term “linear” in reference to a double-stranded DNA molecule described herein, means a nucleic acid comprising two DNA strands or portions of strands which hybridize with each other (thereby forming a double-stranded region), wherein the structure comprises two ends. An end may be a closed end or an open end. The two strands that hybridize with each other may be partially or completely complementary. In some embodiments, a linear double-stranded DNA molecule consists of a single strand of DNA that is circular under denaturing conditions, wherein under physiological conditions a first portion of the strand hybridizes to a second portion of the strand (thereby forming a double-stranded region), and the linear double-stranded DNA molecule comprises a first closed end comprising a first loop and a second closed end comprising a second loop.

As used herein, when two entities are “linked”, the two entities are physically connected by means of one or more covalent or noncovalent bond. In some embodiments, the two entities are directly linked, i.e., an atom of the first entity forms a covalent or noncovalent bond with an atom of the second entity. In some embodiments, the two entities are indirectly linked through a third entity; for example A is linked to C by virtue of A being directly linked to B and B being directly linked to C.

As used herein, the term “maintenance sequence” is a DNA sequence or motif that enables or facilitates retention of a DNA molecule in the nucleus through cell division. A maintenance sequence typically enables replication and/or transcription of a DNA molecule in the nucleus by interacting with proteins that facilitate chromatin looping. An example of a maintenance sequence is a scaffold/matrix attached region (S/MAR element).

As used herein, a “pharmaceutical composition” or “pharmaceutical preparation” is a composition or preparation which is indicated for animal, e.g., human or veterinary pharmaceutical use, for example, non-human animal or human prophylactic or therapeutic use. A pharmaceutical preparation comprises an active agent having a biological effect on a cell or tissue of a subject, e.g., having pharmacological activity or an effect in the mitigation, treatment, or prevention of disease, in combination with a pharmaceutically acceptable excipient or diluent. A pharmaceutical composition also means a finished dosage form or formulation of a prophylactic or therapeutic composition.

As used herein, the terms “peptide,” “polypeptide,” and “protein” are used interchangeably and refer to a compound comprising amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. In some embodiments, a polypeptide comprises a non-canonical amino acid residue.

As used herein, “selectable marker” refers to a DNA sequence that encodes a protein or functional RNA that, when introduced into a host cell (e.g., a bacterial host cell) confers a trait suitable for artificial selection of the host cell. In some embodiments, the artificial selection comprises growth under selective conditions, and the selectable marker promotes survival of the host cell under the selective conditions. In some embodiments, the selective conditions comprise presence of an antibiotic or absence of a nutrient. In some embodiments, the selectable marker results in a visually detectable trait (e.g., color) in the host cell.

As used herein, a “sense strand” of a DNA molecule is a strand which has the same sequence as an mRNA or pre-mRNA which encodes for a functional RNA or protein, and does not serve as a template for transcription. An “antisense strand” of a DNA molecule is a strand that has a sequence complementary to an mRNA or pre-mRNA which encodes for a functional RNA or protein and/or can serve as a template for transcription.

As used herein, the term “double-stranded DNA molecule” or dsDNA molecule means a DNA composition comprising two complementary chains of deoxyribonucleotides that base pair to each other. The two complementary strands may have perfect complementarity or may have one or more mismatches, e.g., forming bulges. Either of the two strands may, in some embodiments, have paired regions of self-complementarity that form intramolecular/intrastrand double stranded motifs in a folded configuration, for example, may form hairpin loops, junctions, bulges or internal loops. In some embodiments, the dsDNA molecule comprises one or two closed ends. In some embodiments, the dsDNA molecule is circular or linear. In some embodiments (e.g., in a dsDNA molecule with closed ends) the two complementary chains of deoxyribonucleotides are covalently linked.

As used herein, “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

### BRIEF DESCRIPTION OF THE DRAWINGS

FIG. 1 is an exemplary chromatogram obtained from performing fast protein liquid chromatography (FPLC), specifically hydrophobic interaction chromatography (HIC), with T5 exonuclease-treated circular dsDNA molecules. X-axis: elution volume (mL). Y-axis: relative absorbance at 260 nm (mAU).

FIG. 2 is an exemplary chromatogram obtained from performing high-performance liquid chromatography (HPLC) on circular dsDNA molecules following enrichment with T5 exonuclease treatment and HIC. X-axis: time (min). Y-axis: relative absorbance at 260 nm (mAU).

FIG. 3 is an exemplary chromatogram obtained from performing HIC with purified circular dsDNA molecules (following T5 exonuclease treatment and HIC). X-axis: elution volume (mL). Y-axis: relative absorbance at 260 nm (mAU).

FIG. 4 is an exemplary chromatogram obtained from performing HIC with T5 exonuclease-treated circular hemi-modified double-stranded DNA (cheDNA) molecules. X-axis: elution volume (mL). Y-axis: relative absorbance at 260 nm (mAU).

FIG. 5 is an exemplary chromatogram obtained from performing HIC with purified cheDNA molecules (following T5 exonuclease treatment and HIC). X-axis: elution volume (mL). Y-axis: relative absorbance at 260 nm (mAU).

### DETAILED DESCRIPTION

This disclosure relates to methods of making a composition comprising DNA molecules, e.g., circular double-stranded DNA (cdsDNA) molecules. This disclosure also provides compositions and methods for providing an effector, e.g., a therapeutic effector, to a cell, tissue or subject, e.g., in vivo or in vitro. The effector may be a DNA sequence, a polypeptide, e.g., a therapeutic protein, or an RNA, e.g., a regulatory RNA or an mRNA.

### Elements of DNA Molecules

The DNA molecules described herein (e.g., dsDNA molecules described herein, e.g., cdsDNA molecules described herein) can contain elements sufficient to deliver an effector sequence to a target cell, tissue or subject. In some embodiments, the effector sequence is a DNA sequence. In some embodiments, the DNA molecule drives expression of an effector, e.g., the DNA molecule comprises a promoter and a sequence encoding an RNA or a polypeptide, e.g., a therapeutic RNA or polypeptide. In some embodiments, the DNA molecules described herein further contain a maintenance sequence.

In some embodiments, the DNA molecule comprises a double-stranded DNA molecule. In some embodiments, the DNA molecule comprises a circular double-stranded DNA molecule, in which the circular double-stranded DNA molecule is a plasmid or a minicircle. In some embodiments, the DNA molecule comprises a circular double-stranded DNA molecule that substantially lacks a material portion of a vector backbone (e.g., plasmid backbone). In some embodiments, the DNA molecule comprises a circular double-stranded DNA molecule that is not supercoiled.

In some embodiments, a DNA molecule disclosed herein (e.g., a cdsDNA molecule disclosed herein) is at least about 10 nucleotides, at least about 15 nucleotides, at least about 20 nucleotides, at least about 25 nucleotides, at least about 30 nucleotides, at least about 40 nucleotides, at least about 50 nucleotides, at least about 60 nucleotides, at least about 70 nucleotides, at least about 75 nucleotides, at least about 80 nucleotides, at least about 90 nucleotides, at least about 100 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 2000 nucleotides, at least about 3000 nucleotides, at least about 4000 nucleotides, at least about 5000 nucleotides, at least about 6000 nucleotides, at least about 7000 nucleotides, at least about 8000 nucleotides, at least about 9000 nucleotides, at least about 10,000 nucleotides, at least about 11,000, or at least about 12,000 nucleotides in length. In some embodiments, a double-stranded DNA molecule is less than 50, less than 60, less than 70, less than 80, less than 90, less than 100, less than 200, less than 300, less than 400, less than 500, less than 600, less than 700, less than 800, less than 900, less than 1000, less than 2000, less than 3000, less than 4000, less than 5000, less than 6000, less than 7000, less than 8000, less than 9000, less than 10,000, less than 11,000, or less than 12,000 nucleotides in length. In some embodiments, the DNA molecule disclosed herein is between 10-15, 15-20, 20-25, 25-30, 20-30, 30-35, 35-40, 30-40, 40-45, 45-50, 40-50, 50-55, 55-60, 60-70, 70-80, 50-75, 75-100, 80-90, 90-100, 100-200, 200-300, 300-400, 400-500, 300-500, 500-600, 600-700, 700-800, 800-900, 900-1000, 500-1000, 1000-2000, 2000-3000, 3000-4000, 4000-5000, 5000-6000, 6000-7000, 7000-8000, 8000-9000, 9000-10,000, 10,000-11,000, or 11,000-12,000 nucleotides in length. In some embodiments, the size of a DNA molecule disclosed herein is a length sufficient to encode useful polypeptides or RNAs.

In some embodiments, a DNA molecule described herein is resistant to endonuclease digestion and/or resistant to immune sensor recognition. In some embodiments, at least 80%, at least 85%, at least 90%, at least 95%, at least 98%, at least 99%, or 100% of the sugars of a DNA molecule described herein are deoxyribose sugars.

In some embodiments, a DNA molecule described herein can be replicated (e.g., by a DNA polymerase native to a cell comprising the DNA molecule). In some embodiments, a DNA molecule described herein cannot be replicated. In some embodiments, a DNA molecule or a portion thereof can be integrated into the genome. In some embodiments, a DNA molecule or a portion thereof cannot be integrated into the genome.

A double-stranded DNA molecule described herein may have less than a threshold level of single stranded structures. In one embodiment, the double-stranded DNA molecule does not comprise more than 20, 18, 16, 14, 12, 10, 8, 7, 5, 4, 3, 2, or 1 single stranded region longer than 100, 80, 70, 60, 50, 40, 30, 20 or 10 bases, e.g., does not comprise single stranded regions longer than 100, 80, 70, 60, 50, 40, 30, 20 or 10 bases.

In some embodiments, a double-stranded DNA molecule described herein comprises a sense strand and an antisense strand.

In some embodiments, a double-stranded DNA molecule described herein can be concatemerized. In some embodiments, a double-stranded DNA molecule described herein cannot be concatemerized.

In some embodiments, a dsDNA molecule described herein is asymmetrically modified, where one strand comprises chemically modified nucleobases and the other strand is substantially free of chemically modified nucleobases. In some embodiments, the hemi-modified DNA molecule may be completely free of chemically modified nucleotides on the antisense strand, and in other embodiments, the hemi-modified DNA molecule may comprise a few chemical modifications (such as backbone modifications, e.g., phosphorothioate) on the antisense strand. In some embodiments, the hemi-modified DNA molecule comprises chemically modified nucleotides (e.g., nucleotides comprising chemically modified nucleobases) on the sense strand.

### Sequence Elements of DNA Molecules

In some embodiments, a DNA molecule described herein (e.g., a dsDNA molecule described herein, e.g., a cdsDNA molecule described herein) comprises a promoter sequence. In some embodiments, a DNA molecule described herein comprises an effector sequence (e.g., a therapeutic effector sequence) operably linked to the promoter sequence. In some embodiments, a DNA molecule described herein comprises a heterologous functional sequence. In some embodiments, a DNA molecule described herein comprises a maintenance sequence. In some embodiments, a DNA molecule described herein comprises an origin of replication. In some embodiments, the DNA molecule comprises one, two, three, four, or all of a promoter sequence, an effector sequence, a heterologous functional sequence, a maintenance sequence, or an origin of replication. In some embodiments, the DNA molecule comprises a promoter sequence, an effector sequence, and a heterologous functional sequence. In some embodiments, the DNA molecule comprises a promoter sequence, an effector sequence, and a maintenance sequence. In some embodiments, the DNA molecule comprises a promoter sequence, an effector sequence, and an origin of replication. In some embodiments, the DNA molecule comprises a promoter sequence, an effector sequence, a heterologous functional sequence, and a maintenance sequence. In some embodiments, the DNA molecule comprises a promoter sequence, an effector sequence, a heterologous functional sequence, and an origin of replication. In some embodiments, the DNA molecule comprises a promoter sequence, an effector sequence, a maintenance sequence, and an origin of replication. In some embodiments, the DNA molecule comprises a promoter sequence, an effector sequence, a heterologous functional sequence, a maintenance sequence, and an origin of replication. In the case of double-stranded DNA, in some embodiments, the promoter and/or effector sequence is in the double-stranded region.

In some embodiments, the effector sequence encodes a polypeptide (e.g., a protein). In some embodiments, the effector sequence encodes a functional RNA (e.g., a miRNA, siRNA, or tRNA). In some embodiments, the effector sequence is heterologous to a target cell.

### Promoters and Other Regulatory Sequences

A DNA molecule described herein (e.g., a cdsDNA molecule described herein) may contain a promoter sequence (a DNA sequence at which RNA polymerase and transcription factors bind to, directly or indirectly, to initiate transcription) operably linked to an effector sequence. A promoter sequence may be found in nature operably linked to the effector sequence, or may be heterologous to the effector sequence. A promoter sequence described herein may be native to the target cell or tissue, or heterologous to the target cell or tissue. A promoter sequence may be constitutive, inducible and/or tissue-specific.

Examples of constitutive promoter sequences include sequences of the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985), the dihydrofolate reductase promoter, the beta-actin promoter, the phosphoglycerol kinase (PGK) promoter, and the EF1alpha promoter.

Inducible promoter sequences allow regulation of expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of sources. Examples of inducible promoter sequences regulated by exogenously supplied promoters include sequences of the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al, Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)).

In some embodiments, the native promoter sequence for the sequence encoding the effector can be used.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoter sequences, enhancer sequences, etc.) are known in the art. Exemplary tissue-specific regulatory sequences include, but are not limited to the following tissue specific promoter sequences: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a alpha-myosin heavy chain (a-MHC) promoter, or a cardiac Troponin T (cTnT) promoter. Other exemplary promoter sequences include sequences of the Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor alpha-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be known to the skilled artisan.

Examples of tissue/cell specific promoter sequences are listed in Table 1:

TABLE 1
Tissue or cell specific promoter sequences

| | | Accession Number; Human |
|---|---|---|
| Tissue/Cell | Promoter | Genome Coordinate (hg38) |
| Skeletal | ACTA1 | NM_001100; chr1: 229,439,090- |
| muscle | | 229,432,090 |
| Melanoma | TYR | NM_000372; chr11: 89,300,750- |
| | | 89,293,750 |
| Hepatoma | a- | NM_001354717; chr4: 73,461,175- |
| | fetoprotein | 73,454,175 |
| Mammary | Mucin 1 | NM_001371720; |
| carcinoma | | chr1: 155,197,900-155,190,900 |
| Prostate | KLK3 | NM_001648; chr19: 50,865,760- |
| Cancer | | 50,858,760 |
| Neuronal | ENO2 | NM_001975; chr12: 6,928,700- |
| cells | | 6,921,700 |
| Response to | HIF- | NM_001530; chr14: 61,753,200- |
| Hypoxia | 1alpha | 61,746,200 |
| Retinoblastoma | E2F1 | NM_005225; chr20: 33,691,380- |
| | | 33,684,380 |
| Ionizing | EGR-1 | NM_001964; chr5: 138,474,303- |
| radiation | | 138,467,303 |
| Oncogene | ErbB2 | NM_004448; chr17: 39,735,530- |
| | | 39,728,530 |
| Endothelial | vWF | NM_000552; chr12: 6,129,670- |
| cells | | 6,122,670 |
| Endothelial | FLT-1 | NM_002019; chr13: 28,500,100- |
| cells | | 28,493,100 |
| Endothelial | ICAM-2 | NM_001099786; |
| cells | | chr17: 64,025,630-64,018,630 |
| Retinal | VMD2 | NM_004183; chr11: 61,972,630- |
| pigment | | 61,965,630 |
| epithelium | | |
| Rod cells | RHO | NM_000539; chr3: 129,540,350- |
| | | 129,533,350 |
| Cone cells | Red/green | NM_020061; chrX: 154,164,030- |
| | opsin | 154,157,030 |
| | (OPN1LW) | |
| Ganglion | Thymocyte | NM_006288; chr11: 119,428,150- |
| cells | antigen | 119,421,150 |
| | (Thy1) | |
| T cells | TIM3 | NM_032782; chr5: 157,114,050- |
| | | 157,107,050 |
| T cells | FOXP3 | NM_014009; chrX: 49,269,700- |
| | | 49,262,700 |
| PBMCs | Vβ6.7 | ENST00000390373.2; |
| | | chr7: 142,493,295-142,486,295 |
| Cell cycle | Cdk1 | NM_001786; chr10: 60,799,850- |
| | | 60,792,850 |

The DNA molecules described herein may also include other native or heterologous expression control elements, such as enhancer elements, polyadenylation sites or Kozak consensus sequences.

### Effector Sequence

The effector sequence of a DNA molecule described herein (e.g., a cdsDNA molecule described herein) may be, e.g., a functional DNA sequence, e.g., a therapeutically functional DNA sequence; a DNA sequence encoding a therapeutic peptide, polypeptide or protein; or a DNA sequence encoding a therapeutic RNA (e.g., a non-coding RNA).

### DNA Effectors:

A therapeutically functional DNA sequence may be a DNA sequence that forms a functional structure, e.g., a DNA sequence comprising a DNA aptamer, DNAzyme or allele-specific oligonucleotide (a DNA ASO). A therapeutically functional DNA sequence typically lacks a promoter operably linked. In embodiments, a DNA molecule described herein may include one or a plurality of functional DNA sequences, e.g., 2, 3, 4, 5, 6, or more sequences, which may be the same or different.

### Polypeptide Effectors:

A DNA sequence encoding a therapeutic polypeptide may be a DNA sequence encoding one or more effector which is a peptide, protein, or combinations thereof. For example, the DNA sequence encodes an mRNA. The peptide or protein may be: a DNA binding protein; an RNA binding protein; a transporter; a transcription factor; a translation factor; a ribosomal protein; a chromatin remodeling factor; an epigenetic modifying factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a CRISPR-linked enzyme, e.g. a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; a protease; an integrase; a recombinase; a topoisomerase; a gyrase; a helicase; a lysosomal acid hydrolase); an antibody (e.g., an intact antibody, a fragment thereof, or a nanobody); a signaling peptide; a receptor ligand; a receptor (e.g., a chimeric antigen receptor (CAR) or a T cell receptor); a clotting factor; a coagulation factor; a structural protein; a caspase; a membrane protein; a mitochondrial protein; a nuclear protein; or an engineered binder such as a centyrin, darpin, or adnectin. See, e.g., Gebauer & Skerra. 2020. Annual Review of Pharmacology and Toxicology 60:1, 391-415.

In embodiments, a DNA molecule described herein may include one or a plurality of sequences encoding a polypeptide, e.g., 2, 3, 4, 5, 6, or more sequences encoding a polypeptide. Each of the plurality may encode the same or different protein. For example, a sequence described herein may include multiple sequences encoding multiple proteins, e.g., a plurality of proteins in a biological pathway.

In some embodiments, a DNA molecule or sequence described herein may include a plurality of sequences encoding a polypeptide, e.g., 2, 3, 4, 5, 6, or more sequences encoding a polypeptide, separated by a self-cleaving peptide, e.g., P2A, T2A, E2A or F2A. self-cleaving peptides are typically 18-22 amino acids long, and can induce ribosomal skipping during protein translation so that two polypeptides can be encoded in the same transcript. Each of the polypeptides may encode the same or different protein. In one embodiment, a DNA molecule or sequence described herein may include a promoter followed by a sequence encoding a first polypeptide of interest, a sequence encoding a 2A self-cleaving peptide, a sequence encoding a second polypeptide of interest, and a polyA site. In another embodiment, a DNA molecule or sequence described herein may include a promoter followed by a sequence encoding the first polypeptide of interest, a first 2A self-cleaving peptide, a second polypeptide of interest, a sequence encoding a second 2A self-cleaving peptide, a sequence encoding a third polypeptide of interest, and a polyA site.

In some embodiments, the effector comprises a cell penetrating polypeptide. In some embodiments, the effector is a fusion protein that comprises a cell penetrating polypeptide and a second amino acid sequence. In some embodiments, the DNA molecule does not comprise a cell penetrating polypeptide. For example, in some embodiments, the DNA molecule does not comprise a fusion protein that comprises a cell penetrating polypeptide.

### RNA Effectors:

An effector sequence may be a DNA sequence encoding a non-coding RNA, e.g., one or more of a short interfering RNA (siRNA), a microRNA (miRNA), long non-coding RNA, a piwi-interacting RNA (piRNA), a small nucleolar RNA (snoRNA), a small Cajal body-specific RNA (scaRNA), a transfer RNA (tRNA), a ribosomal RNA (rRNA), an RNA aptamer, and a small nuclear RNA (snRNA). In some embodiments, a DNA molecule described herein comprises a sequence encoding an RNA (e.g., an mRNA, siRNA, or miRNA). In some embodiments, a DNA molecule described herein does not comprise a sequence encoding an RNA.

In some embodiments, the DNA molecule disclosed herein comprises one or more expression sequences that encode a regulatory RNA, e.g., an RNA that modifies expression of an endogenous gene and/or an exogenous gene. In some embodiments, the DNA molecule disclosed herein can comprise a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA. In one embodiment, the regulatory nucleic acid targets a host gene. A regulatory nucleic acid may include, but is not limited to, a nucleic acid that hybridizes to an endogenous gene, e.g., an antisense RNA, a guide RNA, a nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In one embodiment, the sequence is an miRNA. In some embodiments, the regulatory nucleic acid targets a sense strand of a host gene. In some embodiments, the regulatory nucleic acid targets an antisense strand of a host gene.

In some embodiments, the DNA molecule disclosed herein encodes a guide RNA. Guide RNA sequences are generally designed to have a sequence having a length of between 15-30 nucleotides (e.g., 17, 19, 20, 21, 24 nucleotides) that is complementary to the targeted nucleic acid sequence, and a region that facilitates complex formation (e.g., with a tracrRNA or a nuclease). Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991. The gRNA may recognize specific DNA sequences (e.g., sequences adjacent to or within a promoter, enhancer, silencer, or repressor of a gene). In one embodiment, the gRNA is used as part of a CRISPR system for gene editing. For the purposes of gene editing, the DNA molecule disclosed herein may be designed to include one or multiple sequences encoding guide RNA sequences corresponding to a desired target DNA sequence; see, for example, Cong et al. (2013) Science, 339:819-823; Ran et al. (2013) Nature Protocols, 8:2281-2308.

A DNA molecule disclosed may encode certain regulatory nucleic acids that can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. Such RNAi molecules include, but are not limited to: short interfering RNAs (siRNAs), double-strand RNAs (dsRNA), micro RNAs (miRNAs), short hairpin RNAs (shRNA), meroduplexes, dicer substrates (U.S. Pat. Nos. 8,084,599 8,349,809 and 8,513,207), and RNA antisense oligonucleotides (RNA ASOs).

In one embodiment, the DNA molecule disclosed herein comprises a sequence comprising a sense strand of a lncRNA. In one embodiment, the DNA molecule or sequence disclosed herein comprises a sequence encoding an antisense strand of a lncRNA.

The DNA molecule disclosed herein may encode a regulatory nucleic acid substantially complementary, or fully complementary, to a fragment of an endogenous gene or gene product (e.g., mRNA). The regulatory nucleic acids may complement sequences at the boundary between introns and exons, in between exons, or adjacent to exon, to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid comprises a protein-binding site that can bind to a protein that participates in regulation of expression of an endogenous gene or an exogenous gene.

The length of a DNA molecule disclosed herein that may encode a regulatory nucleic acid that hybridizes to a transcript of interest and may be, for instance, between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

A DNA molecule disclosed herein may encode a micro-RNA (miRNA) molecule identical to about 5 to about 30 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search. In some embodiments, the DNA molecule disclosed herein encodes at least one miRNA, e.g., 2, 3, 4, 5, 6, or more. In some embodiments, the DNA molecule disclosed herein comprises a sequence that encodes an miRNA having at least about 75%, at least about 80%, at least about 85%, at least about 90%, at least about 95%, at least about 96%, at least about 97%, at least about 98%, at least about 99% or 100% nucleotide sequence identity to any one of the nucleotide sequences or a sequence that is complementary to a target sequence. Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed by technologies known in the art. In addition, there are computational tools that increase the chance of finding effective and specific sequence motifs (see, e.g., Lagana et al., Methods Mol. Bio., 2015, 1269:393-412).

The DNA molecule disclosed herein may modulate expression of RNA encoded by a gene. Because multiple genes can share some degree of sequence homology with each other, in some embodiments, the DNA molecule disclosed herein can be designed to target a class of genes with sufficient sequence homology. In some embodiments, the DNA molecule disclosed herein can contain a sequence that has complementarity to sequences that are shared amongst different gene targets or are unique for a specific gene target. In some embodiments, the DNA molecule disclosed herein can be designed to target conserved regions of an RNA sequence having homology between several genes thereby targeting several genes in a gene family (e.g., different gene isoforms, splice variants, mutant genes, etc.). In some embodiments, the DNA molecule disclosed herein can be designed to target a sequence that is unique to a specific RNA sequence of a single gene.

In embodiments, the effector sequence encoding a regulatory RNA has a length less than 5000 bps (e.g., less than about 5000 bps, 4000 bps, 3000 bps, 2000 bps, 1000 bps, 900 bps, 800 bps, 700 bps, 600 bps, 500 bps, 400 bps, 300 bps, 200 bps, 100 bps, 50 bps, 40 bps, 30 bps, 20 bps, 10 bps, or less). In some embodiments, the effector sequence has, independently or in addition to, a length greater than 10 bps (e.g., at least about 10 bps, at least about 20 bps, at least about 30 bps, at least about 40 bps, at least about 50 bps, at least about 60 bps, at least about 70 bps, at least about 80 bps, at least about 90 bps, at least about 100 bps, at least about 200 bps, at least about 300 bps, at least about 400 bps, at least about 500 bps, at least about 600 bps, at least about 700 bps, at least about 800 bps, at least about 900 bps, at least about 1000 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 1.9 kb, at least about 2 kb, at least about 2.1 kb, at least about 2.2 kb, at least about 2.3 kb, at least about 2.4 kb, at least about 2.5 kb, at least about 2.6 kb, at least about 2.7 kb, at least about 2.8 kb, at least about 2.9 kb, at least about 3 kb, at least about 3.1 kb, at least about 3.2 kb, at least about 3.3 kb, at least about 3.4 kb, at least about 3.5 kb, at least about 3.6 kb, at least about 3.7 kb, at least about 3.8 kb, at least about 3.9 kb, at least about 4 kb, at least about 4.1 kb, at least about 4.2 kb, at least about 4.3 kb, at least about 4.4 kb, at least about 4.5 kb, at least about 4.6 kb, at least about 4.7 kb, at least about 4.8 kb, at least about 4.9 kb, at least about 5 kb or greater).

In some embodiments, a DNA molecule disclosed herein comprises one or more of the features described herein, e.g., one or more structural DNA sequence, a sequence encoding one or more peptides or proteins, a sequence encoding one or more regulatory element, a sequence encoding one or more regulatory nucleic acids, e.g., one or more non-coding RNAs, other expression sequences, and any combination of the aforementioned. A construct described herein may have one or a plurality of effector sequences, e.g., 2, 3, 4, 5 or more effector sequences. In the case of a plurality of effector sequences in a single construct, the effector sequences may be the same or different.

In one embodiment, the DNA molecule includes a therapeutically functional, structural DNA sequence. In one embodiment, the DNA molecule includes a promoter and a sequence encoding a therapeutic peptide, polypeptide, or protein described herein. In one embodiment, the DNA molecule includes a promoter and a sequence encoding a regulatory RNA described herein.

In some embodiments, the effector sequence that encodes a polypeptide or protein is codon optimized, e.g., codon optimized for expression in a mammal, e.g., a human. In general, codon optimization means modifying a nucleic acid sequence for enhanced expression in the host cells of interest by replacing at least one codon (e.g., one or more, e.g., 1, 2, 3, 4, 5, 10, 15, 20, 25, 50, or more codons; e.g., at least 1%, at least 5%, at least 10%, at least 20%, at least 25%, at least 50%, at least 60%, at least 70%, at least 80%, at least 90% or 100%) of the native sequence with codons that are more frequently or most frequently used in the genes of that host cell while maintaining the native amino acid sequence. Codon usage tables are available, for example, at the “Codon Usage Database” available at www.kazusa.or.jp/codon. These tables can be adapted in a number of ways, see, e.g., Nakamura et al., 2000, Nucl. Acids Res. 28:292. Computer algorithms for codon optimizing a particular sequence for expression in a particular host cell are also available, such as Gene Forge.

### Maintenance Sequence

A DNA molecule disclosed herein may include a maintenance sequence that supports or enables sustained gene expression through successive rounds of cell division and/or progenitor differentiation in a host cell for a DNA described herein. In embodiments, a maintenance sequence is a nuclear scaffold/matrix attachment region (S/MAR). S/MAR elements are diverse, AT-rich sequences ranging from 60-500 bp that are conserved across species, thought to anchor chromatin to nuclear matrix proteins during interphase (Bode et al. 2003. Chromosome Res 11, 435-445). An S/MAR can be incorporated into a DNA molecule described herein to facilitate long-term transgene expression and extra-chromosomal maintenance. In one embodiment, the maintenance sequence is human interferon-beta MAR (5′tataattcactggaatttttttgtgtgtatggtatgacatatgggtteccttttattttttacatataaatatatttecctgtttttetaaaaaagaaaa agatcatcattttcccattgtaaaatgccatatttttttcataggtcacttacata-3′ (SEQ ID NO: 1)), or a functional sequence having at least 80%, at least 90%, at least 95%, or at least 98% identity thereto. In embodiments, S/MARs useful in the constructs described herein can be found by searching the MARome at bioinfo.net.in/MARome, described also by Narwade et al. 2019. Nucleic Acids Research. Volume 47, Issue 14: 7247-7261.

In embodiments, a DNA molecule described herein is capable of replicating in a mammalian cell, e.g., human cell. In some embodiments, a DNA molecule described herein is maintained in a host cell, tissue or subject through at least one cell division. For example, a DNA molecule described herein is maintained in a host cell, tissue or subject through at least 2, at least 3, at least 4, at least 5, at least 6, at least 7, at least 8, at least 10, at least 15, at least 20, at least 40, at least 50 or more cell divisions. In vitro, cell division may be tracked by flow cytometry or microscopy. In vivo, cell division may be tracked by intravital microscopy.

### Other Elements

A DNA molecule disclosed herein (e.g., a cdsDNA molecule described herein) may also include other control elements operably linked to the effector sequence, e.g., the sequence encoding an effector, in a manner which permits its transport, localization, transcription, translation and/or expression in a target cell, or which promotes its degradation or repression of expression in a non-target cell. As used herein, “operably linked” sequences include both expression control sequences that are contiguous with the sequence encoding the effector and expression control sequences that act in trans or at a distance to control the sequence encoding the effector. The precise nature of regulatory sequences needed for gene expression in host cells may vary between species, tissues or cell types, but in general may include, as necessary, 5′ non-transcribed and 5′ non-translated sequences involved with the initiation of transcription and translation respectively, such as a TATA box, capping sequence, CAAT sequence, enhancer elements and the like. Regulatory sequences may also include enhancer sequences or upstream activator sequences as desired. The constructs described herein may optionally include 5′ leader or signal sequences. In some embodiments, the DNA molecule may comprise a sequence encoding a 5′ untranslated region and/or a sequence encoding a 3′ untranslated region.

The DNA molecule may comprise a non-coding region. In some embodiments, the non-coding region is completely free of predicted ORFs. In some embodiments, the non-coding region does not encode a protein sequence. In some embodiments, the non-coding region is not translated or is not translated at a substantial level.

In some embodiments, a DNA molecule described herein lacks a bacterial origin of replication. In some embodiments, a DNA molecule described herein lacks a selectable marker. In some embodiments, a DNA molecule described herein lacks a bacterial origin of replication and lacks a selectable marker.

In some embodiments, a DNA molecule described herein lacks an antibiotic resistance selectable marker. In some embodiments, the DNA molecule lacks a kanamycin resistance selectable marker. In some embodiments, the DNA molecule lacks a spectinomycin resistance selectable marker. In some embodiments, the DNA molecule lacks a streptomycin resistance selectable marker. In some embodiments, the DNA molecule lacks an ampicillin resistance selectable marker. In some embodiments, the DNA molecule lacks a carbenicillin resistance selectable marker. In some embodiments, the DNA molecule lacks a bleomycin resistance selectable marker. In some embodiments, the DNA molecule lacks an erythromycin resistance selectable marker. In some embodiments, the DNA molecule lacks a polymyxin B resistance selectable marker. In some embodiments, the DNA molecule lacks a tetracycline resistance selectable marker. In some embodiments, the DNA molecule lacks a chloramphenicol resistance selectable marker.

In some embodiments, a DNA molecule described herein lacks a bacterial origin of replication or a functional fragment thereof. In some embodiments, a DNA molecule described herein lacks an oriC or a functional fragment thereof. In some embodiments, a DNA molecule described herein does not substantially undergo replication when introduced into a bacterium.

### Chemically Modified Nucleotides

The DNA molecules described herein (e.g., dsDNA molecules described herein, e.g., cdsDNA molecules described herein) may have chemical modifications of the nucleobases, sugars, and/or the phosphate backbone. While not wishing to be bound by theory, such modifications can be useful for protecting a DNA molecule from degradation (e.g., from exonucleases) or from the immune system of a host tissue or subject. In general, a chemically modified nucleotide has the same base-pairing specificity as the unmodified nucleotide, e.g., a chemically modified adenine “A” can base-pair with thymine “T”. One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, chemical modifications (e.g., one or more modifications) are present in each of the sugar and the internucleoside linkage.

Examples of chemical modifications to a DNA molecule useful in the methods described herein include, e.g., N6-Methyladenosine (m6A, 6 mA); 5-formylcytosine (5-formyl-2′-deoxycytosine, 5fC, f5C); 5-carboxylcytosine (5-carboxyl-2′-deoxycytosine, 5-carboxycytosine, ca5C, 5caC); 5-hydroxymethylcytosine (5-hydroxymethyl-2′-deoxycytosine, 5hmC, hm5C); 5-methyldeoxycytosine (5-methylcytosine; 5-methyl-2′-deoxycytosine; m5dC; 5mC, m5C); 5′-methylcytosine; 3-methylcytosine (m3C); 2′-fluoro-2′deoxynucleoside; 5-glucosylmethylcytosine; 5-methyl pyrimidine; 8-oxoguanine (8-oxoG); phosphorothioate; S and R phsophorothioate linkages; methylthymine; N3′-P5′ Phosphoroamidate (NP); cyclohexane nucleic acid (CeNA); tricyclo-DNA (tcDNA). See, e.g., Pu et al. 2020. An in-vitro DNA 25 phosphorothioate modification reaction. Mol Microbiol. 113: 452-463; Zheng & Sheng. 2021. Synthesis of N4-methylcytidine (m4C) and N4,N4-dimethylcytidine (m42C) modified RNA. Current Protocols, 1, e248; Ohkubo et al. 2021. Chemical synthesis of modified oligonucleotides containing 5′-amino-5′-deoxy-5′-hydroxymethylthymidine residues. Current Protocols, 1, e70; Bao & Xu. 2021. Observation of Z-DNA structure via the synthesis of oligonucleotide DNA 30 containing 8-trifluoromethyl-2-deoxyguanosine. Current Protocols, 1, e28; Skakuj et al. 2020. Automated synthesis and purification of guanidine-backbone oligonucleotides. Current Protocols in Nucleic Acid Chemistry, 81, e110.

In some embodiments, a DNA molecule described herein comprises a chemically modified cytosine nucleobase. In some embodiments, a DNA molecule described herein comprises a chemically modified uracil nucleobase. In some embodiments, a DNA molecule described herein comprises a canonical uracil nucleobase.

In some embodiments, a DNA molecule as described herein may comprise a phosphorothioate-modified nucleotide.

In some embodiments, a DNA molecule described herein may include boranophosphate modified nucleotides, e.g., following the methods in Sergueev and Shaw, 1998, J Am Chem Soc, Volume 120, Issue 37:9417-9427. Briefly, H-phosphonate chain elongation is followed by boronation to substitute a borano group for a nonbridging oxygen in the phosphate backbone. The final sample is purified and analyzed by RP-HPLC to determine stereochemistry of the modification. Boranophosphate modified nucleotides are also commercially available.

In some embodiments, a DNA molecule described herein may include 5-methylcytosine modified nucleotides, e.g., made following the methods in Lin et al, 2002, Mol Cell Biol, Volume 22, Issue 3:704-723. Briefly, cytosine or the sequence containing cytosine is incubated with glutathione S-transferase fusion of wild-type Dnmt3a (GST-3a) protein using unlabeled S-adenosylmethionine (AdoMet). The nucleotides are purified and analyzed by HPLC to determine that the nucleotides are methylated at the correct position. 5-methylcytosine modified nucleotides are also available commercially.

In some embodiments, a DNA molecule described herein may include 7-methylguanine modified nucleotides. In one embodiment, 7-methylguanine modified nucleotides are made following the methods in Jones and Robins, 1963, Purine nucleosides. III. Methylation studies of certain naturally occurring purine nucleosides, J Am Chem Soc, Volume 85:193. Briefly, 2′-deoxyguanosine in dimethyl sulfoxide is treated with methyl iodide. The nucleotides are purified and analyzed by HPLC to determine that the nucleotides are methylated at the correct position. In another embodiment, 7-methylguanine modified nucleotides are made according to the methods described in Hendler et al, 1970, Volume 9, Issue 21:4141:4153, and Kore and Parmar, 2006, Biochemistry, Volume 25, Issue 3:337-340. Briefly, instead of guanosine 5′-diphosphate, guanine 5′-diphosphate in water is added to dimethyl sulfate to yield 7-methyl GDP. The nucleotides are purified and analyzed by HPLC to determine that the nucleotides are methylated at the correct position. 7-methylguanine modified nucleotides are also available commercially.

In some embodiments, a DNA molecule described herein comprises methylation at one or more CpG or GpC dinucleotide.

In some embodiments, a DNA molecule described herein comprises a carboxyl modification or a formyl modification.

In embodiments, a DNA molecule described herein, or one strand (e.g., the sense strand or the antisense strand) of the DNA molecule, comprises between 1-100% chemically modified nucleotides, between 1%-90% chemically modified nucleotides, between 1%-80% chemically modified nucleotides, between 1%-70% chemically modified nucleotides, between 1%-60% chemically modified nucleotides, between 1%-50% chemically modified nucleotides, between 1%-40% chemically modified nucleotides, between 1%-30% chemically modified nucleotides, between 1%-20% chemically modified nucleotides, between 1%-15% chemically modified nucleotides, between 1%-10% chemically modified nucleotides, between 20%-90% chemically modified nucleotides, between 20%-80% chemically modified nucleotides. In embodiments, a DNA molecule described herein, or one strand (e.g., the sense strand or the antisense strand) of the DNA molecule, comprises at least 1% chemically modified nucleotides, at least 5% chemically modified nucleotides; at least 10% chemically modified nucleotides; at least 15% chemically modified nucleotides; at least 20% chemically modified nucleotides; at least 25% chemically modified nucleotides; at least 30% chemically modified nucleotides; at least 40% chemically modified nucleotides; at least 50% chemically modified nucleotides; at least 60% chemically modified nucleotides; at least 70% chemically modified nucleotides; at least 80% chemically modified nucleotides; at least 85% chemically modified nucleotides; at least 90% chemically modified nucleotides; at least 92% chemically modified nucleotides; at least 95% chemically modified nucleotides; or at least 97% chemically modified nucleotides. In embodiments, a DNA molecule described herein, or one strand (e.g., the sense strand or the antisense strand) of the DNA molecule, comprises chemically modified nucleotides at between 0%-100% of each distinct nucleotide, e.g., 0%-100% chemically modified T nucleotides, 0%-100% chemically modified A nucleotides, 0%-100% chemically modified C nucleotides, and 0%-100% chemically modified G nucleotides for each construct. In embodiments, a DNA molecule described herein, or one strand (e.g., the sense strand or the antisense strand) of the double-stranded DNA molecule, comprises chemically modified nucleotides at between 0-100%, 10%-100%, 20%-100%, 30%-100%, 40%-100%, 50%-100%, 60%-100%, 10%-50% of each distinct nucleotide, e.g., between 0-100%, 10%-100%, 20%-100%, 30%-100%, 40%-100%, 50%-100%, 60%-100%, 10%-50% of chemically modified T nucleotides; between 0-100%, 10%-100%, 20%-100%, 30%-100%, 40%-100%, 50%-100%, 60%-100%, 10%-50% of chemically modified A nucleotides; between 0-100%, 10%-100%, 20%-100%, 30%-100%, 40%-100%, 50%-100%, 60%-100%, 10%-50% of chemically modified C nucleotides; or between 0-100%, 10%-100%, 20%-100%, 30%-100%, 40%-100%, 50%-100%, 60%-100%, 10%-50% of chemically modified G nucleotides. For example, a DNA molecule could contain 100% chemically modified T nucleotides, 50% chemically modified A nucleotides, 0% chemically modified C nucleotides, and 25% chemically modified G nucleotides.

In embodiments, chemically modified nucleotides, e.g., modifications described herein, can be introduced in the DNA molecules described herein throughout the entire sequence; within an element of a sequence, e.g., an element described herein; at a 5′- or 3′-end; and/or between the last 10, 8, 6, 5, 4, 3, or 2 nucleotides at the 5′- or 3′-end.

In some embodiments, a double-stranded DNA molecule as described herein comprises chemically modified nucleotides on only one strand. In some embodiments, a double-stranded DNA molecule as described herein comprises chemically modified nucleotides on the antisense strand. In some embodiments, a double-stranded DNA molecule as described herein comprises chemically modified nucleotides on the sense strand.

In some embodiments, a DNA molecule comprises a sense strand and an antisense strand, wherein the antisense strand comprises one or more chemically modified nucleotides. In some embodiments, a DNA molecule comprises a sense strand and an antisense strand, wherein the sense strand does not comprise any chemically modified nucleotides. In some embodiments, a DNA molecule comprises a sense strand and an antisense strand, wherein the sense strand comprises one or more chemically modified nucleotides.

In some embodiments, a double-stranded DNA molecule described herein comprises chemically modified nucleotides on both strands. In certain embodiments, both strands comprise chemical modifications at the same positions (e.g., chemically modified nucleotides on one strand are base-paired with chemically modified nucleotides on the opposite strand, and/or non-chemically modified nucleotides on one strand are base-paired with non-chemically modified nucleotides on the opposite strand). In embodiments, the entirety of both strands are composed of chemically modified nucleotides. In other embodiments, the two strands of a double-stranded DNA molecule as described herein comprise different chemical modification patterns (e.g., one or more chemically modified nucleotides on one strand are base-paired with non-chemically modified nucleotides on the other strand). In embodiments, a double-stranded DNA molecule as described herein comprises one or more double-stranded regions in which both strands are chemically modified, and/or one or more double-stranded regions in which neither strand is chemically modified. In embodiments, a double-stranded DNA molecule as described herein comprises one or more double-stranded regions in which one strand is chemically modified and the other is not.

In some embodiments, a chemically modified DNA molecule described herein exhibits decreased recognition by DNA sensors in a host tissue or subject compared to an unmodified DNA molecule of the same sequence, e.g., at least 10%, at least 20%, at least 30%, at least 40%, at least 50%, at least 60%, at least 70%, at least 80%, at least 90%, at least 95% or more decreased recognition by DNA sensors in a host tissue or subject compared to an unmodified DNA molecule of the same sequence. In some embodiments, a chemically modified DNA molecule described herein exhibits decreased degradation by DNA nucleases compared to an unmodified DNA molecule of the same sequence, e.g., at least 10%, at least 20%, at least 30%, at least 40%, at least 50%, at least 60%, at least 70%, at least 80%, at least 90%, at least 95% or more decreased degradation by DNA nucleases in a host tissue or subject compared to an unmodified DNA molecule. In some embodiments, a chemically modified DNA molecule described herein shows decreased activation of the innate immune system in a target/host tissue or subject compared to an unmodified DNA molecule of the same sequence, e.g., at least 10%, at least 20%, at least 30%, at least 40%, at least 50%, at least 60%, at least 70%, at least 80%, at least 90%, at least 95% or more decreased activation of the innate immune system in a target/host tissue or subject compared to an unmodified DNA molecule of the same sequence.

In some embodiments, a DNA molecule comprising chemically modified nucleotides described herein exhibits any of the following properties in a target/host tissue or subject compared to a DNA molecule of the same sequence that does not comprise chemically modified nucleotides (unmodified dsDNA): increased integration of exogenous construct in genome of target cell; increased retention in a target cell through replication; reduced secondary or tertiary structure formation; reduced interaction with innate immune sensors; reduced interaction with nucleases; enhanced stability; enhanced longevity; reduced toxicity; enhanced delivery; increased expression; increased transport across membranes; increased binding to DNA binding moieties such as nuclear DNA binding proteins, transcription factors, chaperones, DNA polymerases. In embodiments, any of the above listed properties is modulated at least 10%, at least 20%, at least 30%, at least 40%, at least 50%, at least 60%, at least 70%, at least 80%, at least 90%, at least 95% or more in a target/host tissue or subject compared to an unmodified DNA molecule of the same sequence.

In some embodiments, a DNA molecule described herein comprises a chemically modified cytosine nucleobase. In some embodiments, the chemically modified cytosine nucleobase comprises a substitution other than hydrogen at the carbon 5 (C-5) position of the nucleobase. In some embodiments, the chemically modified cytosine nucleobase comprises the structure of Formula I:

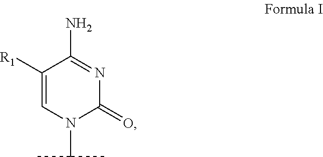

wherein R₁ is selected from the group consisting of —OH; -aldehyde; -carboxylic acid; -alkyl; —(CH₂)ₘOR₂, m=1-3 and R₂═H or a sugar molecule; and -propargylamino. In some embodiments, R₁ is selected from the group consisting of —OH; —CHO; —COOH; -alkyl; —(CH₂)ₘOR₂, m=1-3 and R₂═H or a sugar molecule; and -propargylamino, wherein the alkyl group includes one to six carbons. In some embodiments, R₁ is selected from the group consisting of —OH; —CHO; —COOH; —CH₂OR₃, R₃═H or glucose; -methyl; and -propargylamino. In some embodiments, the chemically modified cytosine nucleobase comprises 5-formylcytosine, 5-hydroxycytosine, 5-carboxycytosine, 5-propargylaminocytosine, 5-methylcytosine, 5-hydroxymethylcytosine, or glucosyl-5-hydroxymethylcytosine. Chemically modified cytosine nucleobases are further described in WO/2024/173836, which is herein incorporated by reference in its entirety.

In some embodiments, a DNA molecule described herein comprises a chemically modified uracil nucleobase. In some embodiments, the chemically modified uracil nucleobase comprises a substitution other than hydrogen or a methyl group at the carbon 5 (C-5) position of the nucleobase. In some embodiments, the chemically modified uracil nucleobase comprises the structure of Formula II:

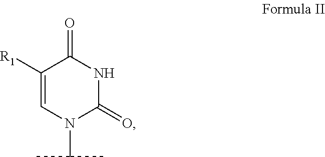

wherein R₁ is selected from the group consisting of —(CH₂)ₘOH, m=1-10; -halogen; —(CH₂)ₙ—CHO, n=0-10; —(CH₂)ₚCOOH, p=0-10; -aminoallyl; —S—(C1-C6)alkyl; and -propargylamino. In some embodiments, R₁ is selected from the group consisting of —(CH₂)ₘOH, m=1-6; -halogen; —(CH₂)ₙ—CHO, n=0-6; —(CH₂)ₚCOOH, p=0-6; -aminoallyl; —S—(C1-C3)alkyl; and -propargylamino. In some embodiments, R₁ is selected from the group consisting of —(CH₂)OH; —I; —Br; —CHO; —COOH; -aminoallyl; —S-methyl; and -propargylamino. In some embodiments, the chemically modified uracil nucleobase comprises 5-hydroxymethyluracil, 5-aminoallyluracil, 5-bromouracil, 5-iodouracil, 5-propargylaminouracil, 5-formyluracil, 5-carboxyuracil, 5-methylthiouracil, or 5-dihydroxypentyluracil. Chemically modified uracil nucleobases are further described in WO/2024/173828, which is herein incorporated by reference in its entirety.

In some embodiments, a DNA molecule described herein comprises a first type of chemically modified nucleobase and a second type of chemically modified nucleobase. In some embodiments, the first type of chemically modified nucleobase is a chemically modified cytosine nucleobase and the second type of chemically modified nucleobase is a chemically modified uracil nucleobase. In some embodiments, the first type of chemically modified nucleobase is a chemically modified cytosine nucleobase and the second type of chemically modified nucleobase is a different chemically modified cytosine nucleobase. In some embodiments, the first type of chemically modified nucleobase is a chemically modified uracil nucleobase and the second type of chemically modified nucleobase is a different chemically modified uracil nucleobase.

### Hydrophobic Interaction Chromatography

In some embodiments, the present disclosure provides methods of making a composition comprising purified DNA molecules described herein, wherein the method comprises performing hydrophobic interaction chromatography (HIC). HIC allows for the purification and separation of biomolecules based on differences in their surface hydrophobicity.

In some embodiments, a method described herein comprises contacting a solution comprising a plurality of DNA molecules described herein to a hydrophobic interaction chromatography (HIC) matrix. In some embodiments, the HIC matrix comprises a butyl (C4) ligand. In some embodiments, the HIC matrix comprises poly(butyl methacrylate-co-ethylene dimethacrylate). In some embodiments, the HIC matrix is comprised by a column. In some embodiments, the column comprises a CIMmultus Monolithic column (Sartorius). The HIC matrix used in the methods described herein can be of various kinds. The matrix may be made of a weakly or strongly hydrophobic material. Thus, in some embodiments, the matrix is substantially hydrophobic. Other hydrophobic matrices comprise synthetic polymeric materials, such as cross-linked polymeric materials. In some embodiments, the matrix is comprised of cross-linked synthetic polymers.

Alternatively, the HIC matrix used in the present method is a hydrophilic carrier coated with hydrophobic ligands. The hydrophilic carrier may be comprised of a cross-linked carbohydrate material, The hydrophobic ligands may be, for example, linear or branched carbon chains, which may be substituted or non-substituted. Alternatively, the ligands may comprise aromatic groups, such as cyclic aromatic groups. The carrier may be of any suitable format, such as porous or non-porous particles, such as irregular or essentially spherical particles; monoliths; membranes, such as stacked membranes; filters; surfaces; capillaries; microtiter plates, etc.

In some embodiments, the matrix is comprised of essentially spherical particles. The methods may be performed with the matrix arranged in the form of an expanded bed or a packed bed, and can be dynamic or run in a batch mode. In packed bed adsorption, the matrix is packed in a chromatographic column and solutions used during a purification process are passed through the column, usually in the same direction. In some embodiments, the matrix is expanded and equilibrated by applying a liquid flow through the column, usually from beneath. A stable fluidised expanded bed is formed when there is a balance between particle sedimentation or rising velocity and the flow velocity during application of the sample and washing steps.

In some embodiments, a HIC matrix described herein is comprised by a column. In some embodiments, the column is a monolithic column. In some embodiments, the column comprises a channel radius of about 2 m. In some embodiments, the column comprises a channel radius of 950 nm-1150 nm. In some embodiments, the column comprises a channel radius of about 1050 nm. In some embodiments, the column has a volume of 1 mL, 4 mL, 8 mL, 40 mL, or 80 mL. In some embodiments, the column has a volume of at least 1 mL, at least 4 mL, at least 8 mL, at least 40 mL, or at least 80 mL. In some embodiments, the column has a volume of 1-5 mL, 5-10 mL, 10-50 mL, or 50-100 mL. In some embodiments, the column is chosen from a Sartorius CIMMULTUS® C4 HLD—1 mL (2 m) column (Catalog no. 311.8136-2), Sartorius CIMMULTUS® C4 HLD—4 mL (2 m) column (Catalog no. 414.8136-2), Sartorius CIMMULTUS® C4 HLD—8 mL (2 m) column (Catalog no. 411.8136-2), Sartorius CIMMULTUS® C4 HLD—40 mL (2 m) column (Catalog no. 614.8136-2), or Sartorius CIMMULTUS® C4 HLD—80 mL (2 m) column (Catalog no. 611.8136-2).

In some embodiments, the solution that is provided (e.g., for contacting to the HIC matrix) is a solution that comprises Tris, ammonium sulfate, and EDTA. In some embodiments, prior to contacting the solution to the HIC matrix, the solution comprises 20-50 mM Tris, 1.0-2.0 M ammonium sulfate, and 5-15 mM EDTA, and wherein the solution has a pH of 7.0-8.0.

In some embodiments, the solution that is provided (e.g., for contacting to the HIC matrix) is a solution that was contacted with a double-stranded DNA exonuclease. In some embodiments, the double-stranded DNA exonuclease initiates exonuclease activity at a 5′ end, 3′ end, or nick. In some embodiments, the double-stranded DNA exonuclease is a T5 exonuclease.

In some embodiments, prior to contacting the solution to the HIC matrix, the HIC matrix is contacted with an equilibration buffer. In some embodiments, the equilibration buffer comprises Tris, ammonium sulfate, and EDTA. In some embodiments, the equilibration buffer comprises 20-50 mM Tris, 1.0-2.0 M ammonium sulfate, and 5-15 mM EDTA. In some embodiments, the equilibration buffer comprises a pH of 7.0-8.0. In some embodiments, the HIC matrix is contacted with 5-20, e.g., 8-12, e.g., about 10 column volumes (CV) of the equilibration buffer. In some embodiments, the HIC matrix is contacted with the equilibration buffer at a 2-10 mL/CV flow rate.

In some embodiments, following contacting the solution to the HIC matrix, cdsDNA molecules are collected from the HIC matrix. In some embodiments, collecting cdsDNA molecules from the HIC matrix comprises collecting a flow-through from the HIC matrix, wherein the flow-through comprises cdsDNA molecules. In some embodiments, the method comprises contacting the collected cdsDNA molecules with a solution comprising sodium citrate. In some embodiments, the solution comprising the collected cdsDNA molecules and sodium citrate comprises 0.5 mM to 2 mM sodium citrate (e.g., 1 mM sodium citrate). In some embodiments, the solution comprising the collected cdsDNA molecules and sodium citrate has a pH between 5.5 and 7.0 (e.g., between 6.0 and 6.5).

In some embodiments, the method comprises contacting the HIC matrix with an elution buffer, wherein the elution buffer comprises one or more impurities, e.g., an impurity described herein. In some embodiments, the method comprises washing the HIC matrix. In some embodiments, the method further comprises contacting the HIC matrix with a second solution comprising a plurality of DNA molecules and repeating one or more steps of the method.

### Production

In some embodiments, a circular hemi-modified dsDNA molecule may be produced as follows. First, a linear dsDNA molecule comprising a first strand (e.g., a sense strand) and a second strand (e.g., antisense strand) is provided, e.g., using routine methods. The second strand may comprise a backbone modification that is resistant to exonuclease activity, e.g., one or more (e.g., two, three, four, five, six, seven, or eight) phosphorothioate linkages. The backbone modifications may be placed at one end of the second strand, in order to protect that strand from exonuclease degradation starting from that end. The linear dsDNA molecule may be incubated with one or more nucleases, e.g., one or more exonucleases, e.g., an exonuclease that acts in a 5′ to 3′ direction (e.g., T7 exonuclease or Lambda Exonuclease) or an exonuclease that acts in a 3′ to 5′ direction, to remove the first strand, thereby producing a single stranded DNA (ssDNA) intermediate. Without wishing to be bound by theory, the backbone modifications on the second strand may protect the second strand from exonuclease degradation, e.g., modification at the 5′ end would protect the strand from an exonuclease that acts in a 5′ to 3′ direction. The ssDNA intermediate may be enriched through treatment of a restriction enzyme, e.g., Mlyl endonuclease, to remove residual linear dsDNA molecules. The ssDNA intermediate may be used to produce a linear, hemi-modified dsDNA in an isothermal extension reaction, e.g., the ssDNA intermediate may be contacted with a polymerase (e.g., a heat activated KME polymerase), unmodified deoxyribose triphosphates, chemically modified nucleotides, and a primer optionally comprising unmodified nucleotides, thereby producing a linear, hemi-modified dsDNA in which the first strand comprises chemically modified nucleotides. The linear, hemi-modified dsDNA may be digested using a restriction enzyme and ligated (e.g., through incubation with a DNA ligase, e.g., T3 Ligase) to produce circular, hemi-modified dsDNA. The circular hemi-modified dsDNA may be enriched through incubation with an exonuclease, e.g., T5 exonuclease, to remove unligated DNA.

In some embodiments, a circular double-stranded DNA (cdsDNA) molecule may be produced as follows. First, a linear dsDNA molecule is provided, e.g., using a polymerase chain reaction (PCR) or other routine methods. The linear dsDNA molecule may contacted with a restriction enzyme (e.g., BsaI), e.g., for 4 to 16 hours (e.g., for 12 to 16 hours), e.g., at about 37° C., e.g., with about 80 rpm agitation. In some embodiments, the restriction enzyme is heat-inactivated, and in other embodiments, the restriction enzyme is not heat-inactivated. The digested DNA molecule may be contacted with a DNA ligase (e.g., a T3 DNA ligase), e.g., in a bioreactor, e.g., with about 80 rpm agitation, e.g., at 23° C.+/−2° C., to form cdsDNA molecules. The DNA ligase may be heat-inactivated, e.g., at 60° C. to 70° C. (e.g., about 65° C.), e.g., for about 1 or 3 hours. The cdsDNA molecule may be concentrated. The buffer may be exchanged using a 10-50 kDa molecular weight cut-off (MWCO) hollow fiber, e.g., with about 5 or 7 diafiltration volumes of water.

In some embodiments, the method comprises making or manufacturing a DNA molecule, the method comprising (a) providing a DNA molecule described herein, and (b) determining whether the structure of the DNA molecule matches a reference structure, thereby making or manufacturing the DNA molecule. In some embodiments, the determining of (b) comprises sequencing the DNA molecule. In some embodiments, the determining of (b) comprises digesting the DNA molecule with a restriction enzyme. In some embodiments, the structure of the DNA molecule that matches the reference structure is identical to the reference structure. In some embodiments, the structure of the DNA molecule that matches the reference structure has the same sequence as the reference structure. In some embodiments, the structure of the DNA molecule that matches the reference structure has the same length as the reference structure.

The DNA molecule may be enriched or purified from impurities or byproducts selected from the group consisting of: endotoxin, mononucleotides, chemically modified mononucleotides, single stranded DNA, linear DNA, concatemeric DNA, proteins (e.g., enzymes, e.g., ligases, restriction enzymes), DNA fragments or truncations. In some embodiments, the purified DNA molecule is substantially free of process byproducts and impurities, e.g., process byproducts or impurities described herein.

In some embodiments, a DNA molecule is formulated with a lipid based carrier, e.g., a lipid nanoparticle (LNP).

The DNA molecule may be sequenced to confirm the desired, designed sequence. In embodiments, other structural analysis of the DNA molecule (e.g., restriction enzyme analysis) may be performed to confirm or verify its sequence.

### Enrichment

A composition described herein (e.g., a composition comprising a dsDNA molecule described herein, e.g., a composition comprising a cdsDNA molecule described herein) is typically enriched to remove process impurities and/or contaminants. In some embodiments, a composition comprising a DNA molecule described herein is enriched. For instance, in some embodiments, at least 60%, at least 65%, at least 70%, at least 75%, or at least 80% by mass of total DNA in the composition may be the DNA molecule (e.g., the circular dsDNA molecule). As an example, the composition may also comprise other forms of DNA, e.g., as a process impurity, for instance host cell DNA. As an example, the composition may comprise a contaminant, such as bacterial or viral or fungal agents.

In some embodiments, a composition described herein (e.g., a composition comprising a DNA molecule, e.g., a pharmaceutical composition comprising a DNA molecule, or a manufacturing intermediate) is free of or is substantially free of one or more process impurity or contaminant, e.g., as described in this section. In some embodiments, a method described herein results in a composition that is free of or is substantially free of one or more process impurity or contaminant, e.g., as described in this section. In some embodiments, a method described herein comprises a step of assaying for one or more process impurity or contaminant, e.g., as described in this section. In some embodiments, the method comprises approving or releasing a batch if the batch is free of or substantially free of the process impurity or contaminant or meets a release criterion for that process impurity or contaminant.

In some embodiments, the process impurity comprises a nonhuman animal serum (e.g., fetal bovine serum); an enzyme, e.g., a ligase, a polymerase, or a digestive enzyme (e.g., a trypsin, a collagenase, a DNase, a RNase, an exonuclease, or an endonuclease, e.g., a restriction endonuclease); a growth factor; a cytokine; an antibody (e.g., a monoclonal antibody); a bead (e.g., an antibody-coated bead); an antibiotic; a cell culture medium; a component of a cell culture medium; a detergent; a protein, e.g., a host cell protein; an extraneous nucleic acid sequence (e.g., a mononucleotide (e.g., a modified mononucleotide), or a DNA fragment or truncation); helper virus contaminant (e.g., infectious virus, viral DNA, or viral proteins); a solvent; a cellular debris; a cell; a pyrogen; a fungus; or any combination thereof, or a portion of any of the foregoing. In some embodiments, the contaminant was a component introduced during a manufacturing process. In some embodiments, the contaminant comprises a viral protein.

In some embodiments, the contaminant comprises an agent for transmissible spongiform encephalopathy (TSE). In some embodiments, a test for this contaminant is performed on a composition for which a bovine material was used in manufacturing.

In some embodiments, the contaminant comprises a zoonotic virus, a porcine circovirus 1, a porcine circovirus 2, or a porcine parvovirus; or any combination thereof, or a portion of any of the foregoing. In some embodiments, a test for this contaminant is performed on a composition for which non-human animal material, e.g., a porcine material, was used in manufacturing.

In some embodiments, the contaminant comprises a virus or portion thereof, e.g., a human virus; human immunodeficiency virus (HIV); HIV-1; HIV-2; hepatitis B virus (HBV); hepatitis C virus (HCV); human TSE, including Creutzfeldt-Jakob disease (CJD); variant CID (vCJD); Treponema pallidum (syphilis); human T-lymphotropic virus (HTLV), HTLV-1, HTLV-2; or cytomegalovirus, human herpesvirus (e.g., human herpesvirus-6, -7 or -8 (HHV-6, -7, or -8)), JC virus, BK virus, Epstein-Barr virus (EBV), human parvovirus B19, human papillomavirus (HPV); an adenovirus, e.g., adenovirus E1; SV40 Large T antigen sequence; HPV E6 or E7 DNA; or any combination thereof, or a portion of any of the foregoing. In some embodiments, a test for this contaminant is performed on a composition for which human donor cells (e.g., leukocyte-rich cells) were used in manufacturing. In some embodiments, a test for this contaminant is performed on a cell bank.

In some embodiments, the contaminant comprises a microbe or a portion thereof, a bacterium (e.g., a Gram-negative bacterium); mycoplasma; spiroplasma (e.g., when insect cells are used); bacterial toxin (e.g., endotoxin); or an adventitious agent, e.g., an adventitious viral agent or a non-viral adventitious agent, or any combination thereof, or a portion of any of the foregoing. In some embodiments, the contaminant comprises a simian virus, e.g., simian polyomavirus SV40 or simian retrovirus, or any combination thereof, or a portion of any of the foregoing. In some embodiments, the contaminant comprises an arbovirus. In some embodiments, the contaminant comprises a bacteriophage. In some embodiments, a test for this contaminant is performed on a cell bank, e.g., a cell bank of bacterial cells.

In some embodiments, the contaminant or process impurity comprises DNA from a host cell, e.g., wherein the host cell is a non-tumorigenic cell. In some embodiments, the DNA is present at a level of less than 10 ng/dose. In some embodiments, the DNA size is below about 200 nucleotides in length.

In some embodiments, the contaminant is an endotoxin. In some embodiments, a level of the endotoxin is less than 5 Endotoxin Unit (EU)/kg body weight/hour, e.g., wherein the composition is formulated for parenteral administration. In some embodiments, a level of the endotoxin is less than 0.2 EU/kg body weight/hour, e.g., wherein the composition is formulated for intrathecal administration. In some embodiments, a level of the endotoxin is not more than 2.0 EU/dose/eye, e.g., wherein the composition is formulated for injection or implantation into the eye, or not more than 0.5 EU/mL, e.g., wherein the composition is formulated for intraocular administration.

In some embodiments, a process impurity comprises an organic solvent, e.g., an aromatic organic solvent, e.g., phenol or chloroform.

In some embodiments, the contaminant or process impurity is described in Chemistry, Manufacturing, and Control (CMC) Information for Human Gene Therapy Investigational New Drug Applications (INDs)—Guidance for Industry (U.S. Department of Health and Human Services, Food and Drug Administration, Center for Biologics Evaluation and Research, January 2020), which is herein incorporated by reference in its entirety.

In some embodiments, the composition is substantially free of (e.g., is free of) a polymerase. In some embodiments, the composition is substantially free of (e.g., is free of) lipids, e.g., LNPs. In some embodiments, the composition is substantially free of (e.g., is free of) nanoparticles.

In some embodiments, the composition is substantially free of (e.g., is free of) agarose. In some embodiments, the composition is substantially free of (e.g., is free of) acrylamide.

In some embodiments, the composition is substantially free of (e.g., is free of) polypeptides.

In some embodiments, a composition (e.g., a pharmaceutical composition) described herein comprises purified cdsDNA molecules, wherein the cdsDNA molecules lack one or both of a bacterial origin of replication and a selectable marker, and wherein 85%-90%, 90%-95%, 95%-96%, 96%-97%, 97%-98%, 98%-99%, or 99%-99.6% by mass of total DNA molecules in the composition are the cdsDNA molecules.

### Pharmaceutical Compositions

The present disclosure includes a DNA molecule described herein (e.g., a cdsDNA molecule described herein) and related compositions in combination with one or more pharmaceutically acceptable excipients and/or carriers.

Pharmaceutical compositions may optionally comprise one or more additional active substances, e.g., therapeutically and/or prophylactically active substances. Pharmaceutical compositions of the present invention are generally sterile and/or pyrogen-free.

A DNA molecule described herein may be formulated without a carrier, e.g., the DNA molecule described herein may be administered to a host cell, tissue or subject “naked”. A naked formulation may include pharmaceutical excipients or diluents but lacks a carrier.

Pharmaceutically acceptable excipients or diluents may comprise an inactive substance that serves as a vehicle or medium for the compositions described herein, such as any one of the inactive ingredients approved by the United States Food and Drug Administration (FDA) and listed in the Inactive Ingredient Database, which is incorporated by reference herein. Non-limiting examples of pharmaceutically acceptable excipients or diluents include solvents, aqueous solvents, non-aqueous solvents, tonicity agents, dispersion media, cryoprotectants, diluents, suspension aids, surface active agents, isotonic agents, thickening agents, emulsifying agents, preservatives, hyaluronidases, dispersing agents, preservatives, lubricants, granulating agents, disintegrating agents, binding agents, antioxidants, buffering agents (e.g., phosphate buffered saline (PBS)), lubricating agents, oils, and mixtures thereof.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

### Carriers

A DNA molecule described herein may also be formulated, or included, with a carrier. General considerations of carriers and delivery of pharmaceutical agents may be found, for example, in “Delivery Technologies for Biopharmaceuticals: Peptides, Proteins, Nucleic Acids and Vaccines” (Lene Jorgensen and Hanne Morck Nielson, Eds.) Wiley; 1st edition (Dec. 21, 2009); and Vargason et al. 2021. Nat Biomed Eng 5, 951-967.

Non-limiting examples of carriers include carbohydrate carriers (e.g., an anhydride-modified phytoglycogen or glycogen-type material, GalNAc), nanoparticles (e.g., a nanoparticle that encapsulates or is covalently linked to the DNA molecule, gold nanoparticles, silica nanoparticles), lipid particles (e.g., liposomes, lipid nanoparticles), cationic carriers (e.g., a cationic lipopolymer or transfection reagent), fusosomes, non-nucleated cells (e.g., ex vivo differentiated reticulocytes), nucleated cells, exosomes, protein carriers (e.g., a protein covalently linked to the DNA molecule), peptides (e.g., cell-penetrating peptides), materials (e.g., graphene oxide), single pure lipids (e.g., cholesterol), or DNA origami (e.g., DNA tetrahedron).

In one embodiment, the DNA molecules, compositions, constructs and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for an agent (e.g., a DNA molecule) described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the DNA molecules described herein.

### Lipid Nanoformulations Lipid-Based Carriers

In some embodiments, compounds, e.g., DNA molecules, described herein are formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more compounds described herein, or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more compounds described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

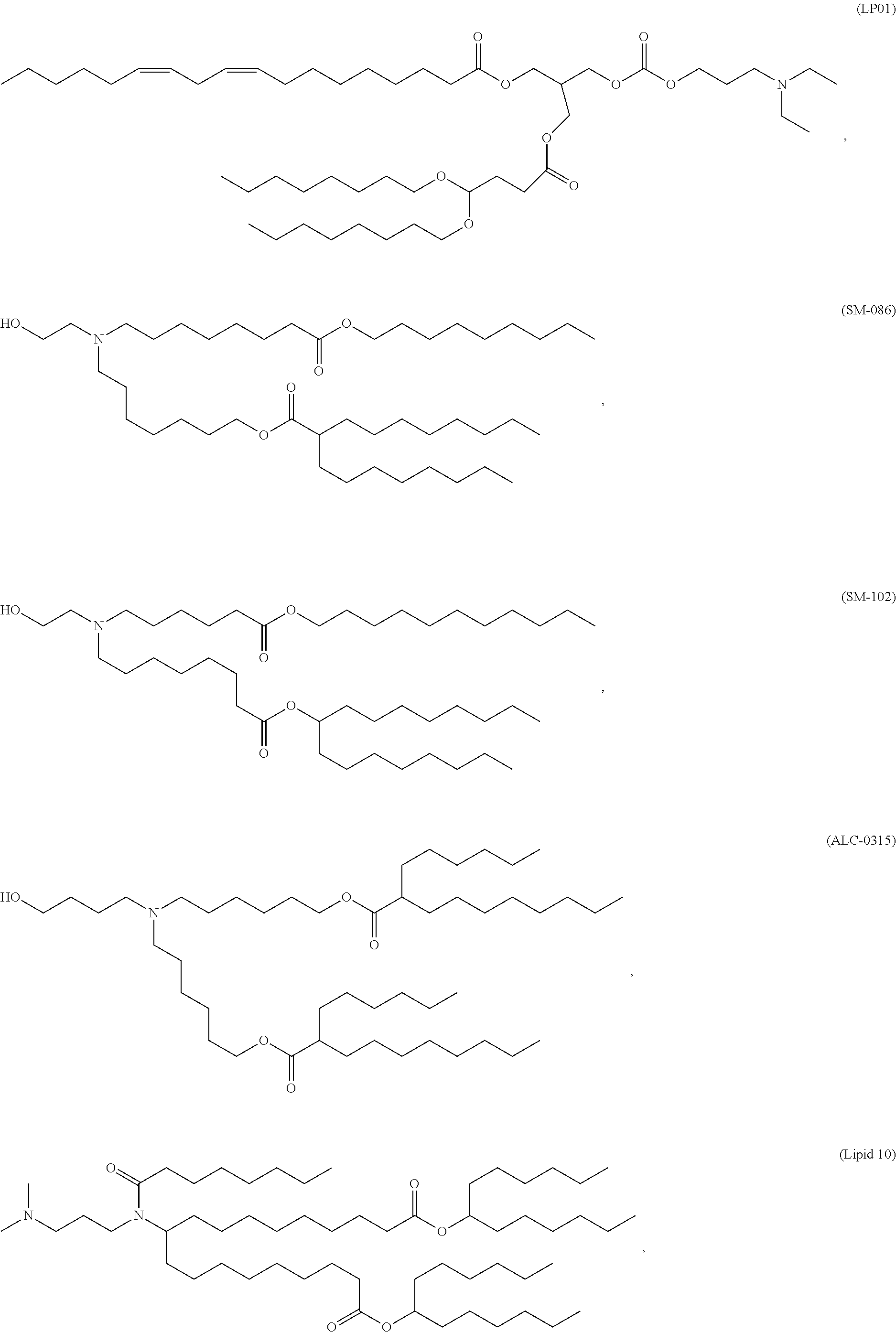

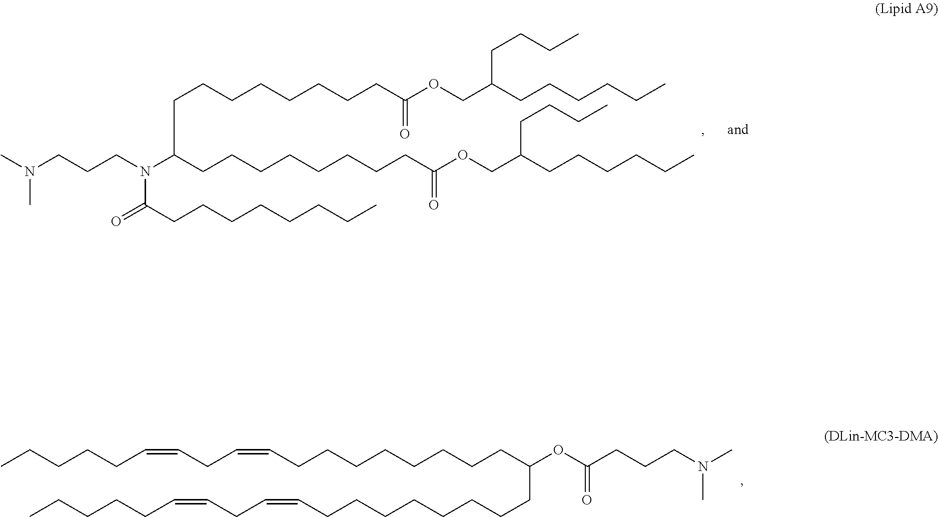

(see WO 2017/004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et al. Nat Rev Mater 6, 1078-1094 (2021). doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y—Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; 111-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

### Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, which is incorporated herein by reference. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipids in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20(6):4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### Polymers and Polyethylene Glycol (PEG)—Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-l-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

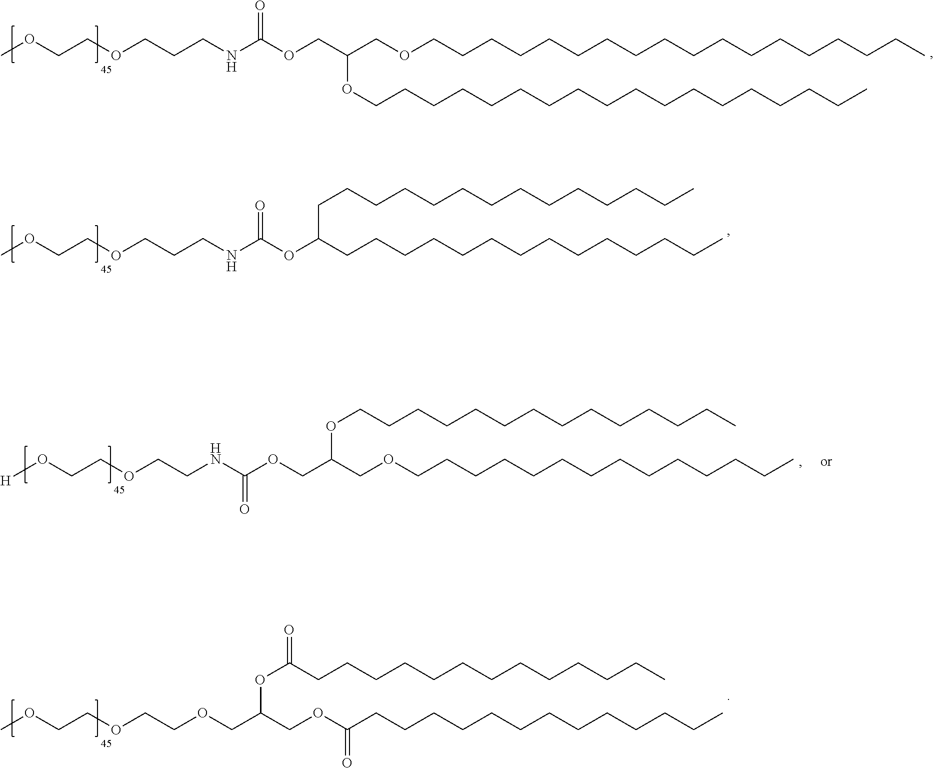

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), which are incorporated herein by reference in their entirety.

### Percentages of Lipid Nanoformulation Components

In some embodiments, the lipid-based carrier (or lipid nanoformulation) comprises one of more of the compounds described herein, optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises a payload (e.g., a DNA molecule described herein). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a payload (e.g., a DNA molecule described herein) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the payload may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a payload (e.g., a DNA molecule described herein) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the payload may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a payload (e.g., a DNA molecule described herein) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the payload may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a payload (e.g., a DNA molecule described herein) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the payload may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a payload (e.g., a DNA molecule described in) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the payload may be at least 70%.

In some embodiments, the lipid-based carrier (or lipid nanoformulation) comprises (i) a DNA molecule; (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a DNA molecule; (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the payload (e.g., an encapsulated therapeutic agent such as a DNA molecule) can be varied as desired. For example, the total lipid components to the payload (mass or weight) ratio can be from about 10:1 to about 30:1. In some embodiments, the total lipid components to the payload ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the payload can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of a payload such as a DNA molecule, describes the amount of the DNA molecule that is encapsulated or otherwise associated with a lipid nanoformulation (e.g., liposome or LNP) after preparation, relative to the initial amount provided. The encapsulation efficiency is desirably high (e.g., at least 70%, 80%, 90%, 95%, or close to 100%). The encapsulation efficiency may be measured, for example, by comparing the amount of DNA molecule in a solution containing the liposome or LNP before and after breaking up the liposome or LNP with one or more organic solvents or detergents. An anion exchange resin may be used to measure the amount of DNA molecule in a solution. Fluorescence may be used to measure the amount of DNA molecule in a solution. For the lipid-based carrier (or lipid nanoformulation) described herein, the encapsulation efficiency of a DNA molecule may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### Route of Administration

A DNA molecule described herein (e.g., a cdsDNA molecule described herein) is introduced into a cell, tissue or subject by any suitable route.

Administration to a target cell or tissue (e.g., ex vivo) may be by methods known in the art such as transfection, e.g., transient or stable transfection using reagents (e.g., liposomal, calcium phosphate) or physical means (e.g., electroporation, gene gun, microinjection, microfluidic fluid shear, cell squeezing). Other methods are described, e.g., in Rad et al. 2021. Adv. Mater. 33:2005363, which is incorporated herein by reference.

Administration to a subject, e.g., a mammal, e.g., a human subject, may be by parenteral (e.g., intravenous, intramuscular, intraperitoneal, subcutaneous, or intracranial) route; by topical administration, transdermal administration or transcutaneous administration. Other suitable routes include oral, rectal, transmucosal, intranasal, inhalation (e.g., via an aerosol), buccal (e.g., sublingual), vaginal, intrathecal, intraocular, transdermal, intraendothelial, in utero (or in ovo), intrapleural, intracerebral, intraarticular, topical, intralymphatic. Also included is direct tissue or organ injection (e.g., to liver, eye, skeletal muscle, cardiac muscle, diaphragm, muscle or brain).

### Applications

A DNA molecule described herein (e.g., a cdsDNA molecule described herein) can be used in therapeutic or health applications for a subject, e.g., a human or non-human animal. Although the descriptions of pharmaceutical compositions provided herein are principally directed to pharmaceutical compositions which are suitable for administration to humans, it will be understood by the skilled artisan that such compositions are generally suitable for administration to any other animal. The subject can be any animal, e.g., a mammal, e.g., a human or non-human mammal. In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk.

In some embodiments, a DNA molecule described herein is provided at a dose of about 0.1-100 mg/kg of DNA.

In some embodiments, a DNA molecule described herein imparts a biological effect of the effector, e.g., expression of a therapeutic polypeptide, on a host cell, tissue or subject over a time period of at least 2, at least 3, at least 4, at least 5, at least 6 days or at least a week; at least 8, at least 9, at least 10, at least 12, at least 14 days or at least two weeks; at least 16, at least 18, at least 20 days or at least 3 weeks; at least 22, at least 24, at least 25, at least 27, at least 28 days or at least a month; at least 2 months, at least 3 months, at least 4 months, at least 5 months, at least 6 months or more; between one week and 6 months, between 1 month to 6 months, between 3 months to 6 months.

In some embodiments, a DNA molecule described herein imparts a biological effect of the effector, e.g., expression of a therapeutic polypeptide, on a host cell, tissue or subject over a time period of at least 1 cell divisions of the host cell.

In embodiments, a DNA molecule described herein can be used to deliver an effector, e.g., an effector described herein, to a cell, tissue or subject.

In embodiments, a DNA molecule described herein can be used to modulate (e.g., increase or decrease) a biological parameter in a cell, tissue or subject. The biological parameter may be an increase or decrease in gene expression of a subject gene in a target cell, tissue or subject. In some embodiments, a DNA molecule described herein increases or decreases a biological activity in a target cell, wherein the biological activity comprises cell growth, cell metabolism, cell signaling, cell movement, specialization, interactions, division, transport, homeostasis, osmosis, or diffusion. In some embodiments, the cell is an animal cell, e.g., a mammalian cell, e.g., a human cell.

In embodiments, a DNA molecule described herein can be used to treat a cell, tissue or subject in need thereof by administering the DNA molecule described herein to such cell, tissue or subject.

In embodiments, the DNA molecule delivers an effector to a cell.

### EXAMPLES

### Example 1: Preparatory Scale Separation of Circular Double-Stranded DNA (cdsDNA) Molecules from Linear and Concatemer Species

This Example demonstrates enrichment of circular double-stranded DNA (cdsDNA) molecules from impurities, e.g., linear and concatemeric DNA molecules.

The synthesis of 1-5 kilobase (kb) cdsDNA molecules began with 4 to 16 hours of digestion at 37° C. of PCR product with a specific restriction enzyme (e.g., BsaI) corresponding to a restriction enzyme recognition sequence in the PCR product. The enzyme was then heat-inactivated, and the digested DNA molecule was circularized using T3 DNA ligase (M0317, New England Biolabs) in a bioreactor at a 100-rpm agitation at room temperature (23° C.+/−2° C.) to form cdsDNA molecules. T3 ligase was heat-inactivated at 65° C. for 1 hour. The ligated DNA molecule was concentrated, and the buffer was exchanged using a 10-50 kDa molecular weight cut off (MWCO) hollow fiber with 5 diafiltration volumes of water.

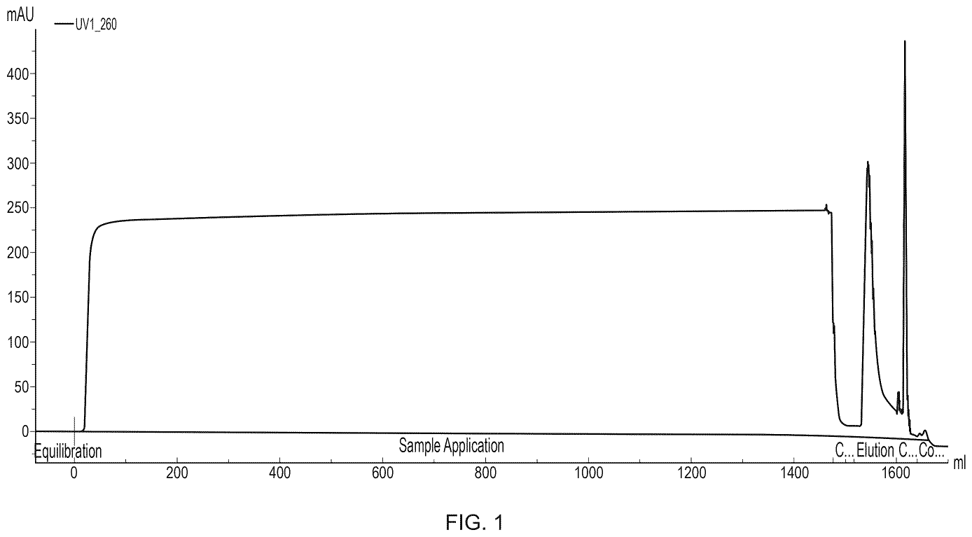

The cdsDNA molecules were enriched from linear DNA molecules and linear concatemer byproduct impurities from the ligation reaction using T5 exonuclease treatment followed by enrichment using fast protein liquid chromatography (FPLC), specifically hydrophobic interaction chromatography (HIC). The linear DNA species were digested with 0.1-2.0 U/μg of T5 exonuclease enzyme at 37° C. with 1×NEB 4 Buffer and 100-300 mM Tris pH 10. The cdsDNA molecules were then purified using an AKTA Pure or AKTA Avant (Cytiva) with CIMmultus C4 HLD 1 mL hydrophobic interaction chromatography monolith columns (Bioseparation/Sartorius) (FIG. 1).

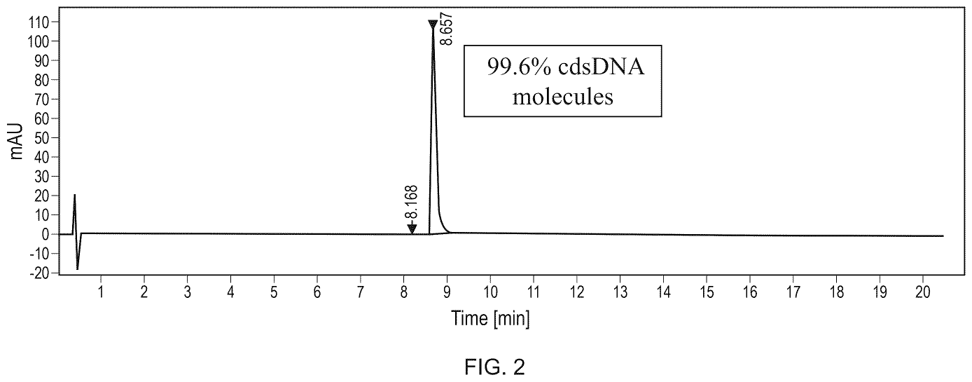

The DNA sample buffer was adjusted after T5 exonuclease treatment to match the equilibration/binding buffer for HIC (20-50 mM Tris, 1.0-2.0 M Ammonium sulfate, 5-15 mM EDTA, pH 7.0-8.0). The column was equilibrated with 10 column volume (CV) of the equilibration/binding buffer at a 2-10 ml/CV flow rate. The T5 exonuclease-treated sample was loaded into the column via the sample pump, and the flow-through containing the circular DNA molecules was collected via Outlet 1. The flow-through material was filtered through a 0.22-0.45 m filter and concentrated in a Repligen system using a 10-50 kDa MWCO hollow fiber followed by 10 diafiltration volume into 1 mM sodium citrate pH 6.0-6.5. The purified circular dsDNA concentration was analyzed by nanodrop and high-performance liquid chromatography (HPLC). Over 90% purity (99.6%) of cdsDNA molecules was achieved with the described method (FIG. 2).

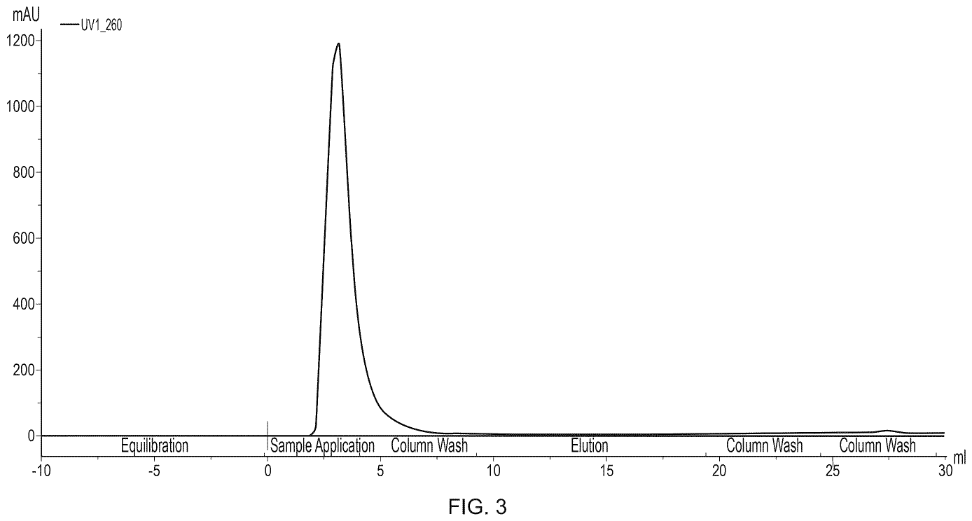

To determine the recovery of the DNA sample, 320 g of purified cdsDNA molecules (following T5 exonuclease treatment and HIC) was injected into a HIC column. FIG. 3 shows the chromatogram of the purified circular dsDNA, in which the peak was only seen on the binding or sample application steps. Table 2 shows the amount injected and recovered from the column post-buffer exchanged, demonstrating that greater than 70% recovery of material post-chromatography and buffer exchange was achieved.

TABLE 2
Recovery of cdsDNA molecules using HIC.

| | Concentration | | | Volume | Yield | Recovery |
|---|---|---|---|---|---|---|
| | (ng/μL) | A260/280 | A260/230 | (μL) | (μg) | (%) |
| DNA pre-HIC | 100.1 | 1.88 | 2.05 | 3200 | 320 | n/a |
| DNA post-HIC | 271.8 | 1.92 | 2.02 | 870 | 236 | 74 |

### Example 2: Preparatory Scale Separation of Circular Hemi-Modified Double-Stranded DNA (cheDNA) from Linear and Concatemer Species

This Example demonstrates enrichment of circular hemi-modified double-stranded DNA (cheDNA) molecules from linear and concatemeric DNA molecules. The cheDNA molecules comprise chemically modified nucleotides (specifically, having chemically modified nucleobases) in one strand, e.g., sense strand, of the dsDNA molecule.

The 1-5 kb cheDNA molecules used in this experiment had an antisense strand that was free of chemically modified nucleobases and a sense strand comprising A, C, G, and either (i) 2′-deoxyuridine or (ii) 5-hydroxymethyl-2′-deoxyuridine. In other words, the cheDNA lacked thymine and instead comprised canonical uracil or 5-hydroxymethyluracil in the sense strand.

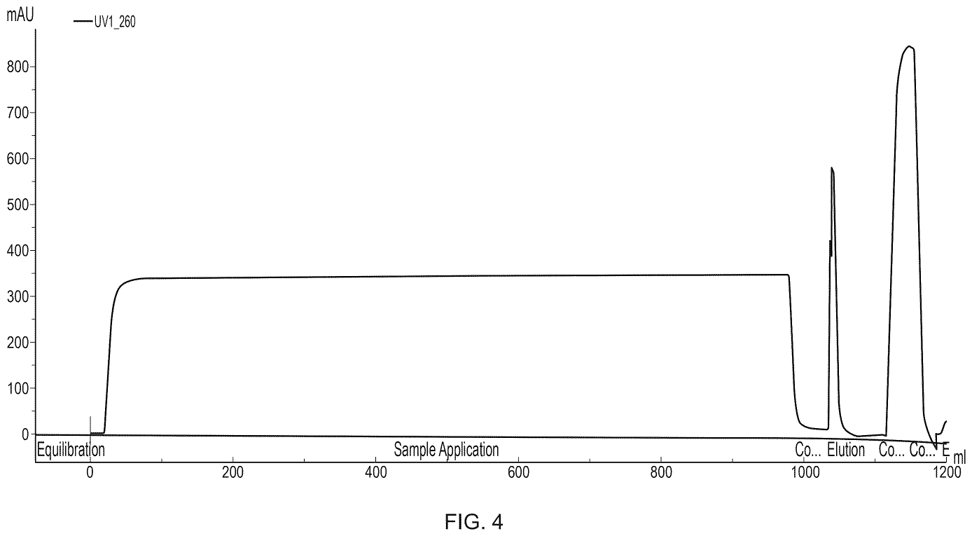

The cheDNA molecules were enriched from linear DNA and linear concatemer byproduct impurities from the ligation reaction using T5 exonuclease treatment followed by enrichment using FPLC, specifically HIC. The linear DNA species were digested with 0.1-2.0 U/μg of T5 exonuclease enzyme at 37° C. with 1×NEB 4 Buffer and 100-300 mM Tris pH 10. The cheDNA molecules were then purified using an AKTA Pure or AKTA Avant (Cytiva) with CIMmultus C4 HLD 1 mL hydrophobic interaction chromatography monolith columns (Bioseparation/Sartorius) (FIG. 4).

The DNA sample buffer was adjusted after T5 exonuclease treatment to match the equilibration/binding buffer for HIC (20-50 mM Tris, 1.0-2.0 M Ammonium sulfate, 5-15 mM EDTA, pH 7.0-8.0). The column was equilibrated with 10 column volumes (CV) of the equilibration/binding buffer at a 2-10 mL/CV flow rate. The T5 exonuclease-treated sample was loaded into the column via the sample pump, and the flow-through containing the circular hemi-modified DNA molecules was collected via Outlet 1. The flow-through material was filtered through a 0.22-0.45 m filter and concentrated in a Repligen system using a 10-50 kDa MWCO hollow fiber followed by 10 diafiltration volumes into 1 mM sodium citrate pH 6.0-6.5. The purified circular hemi-modified dsDNA concentration was analyzed by nanodrop and HPLC. Over 90% purity (99.6%) of cheDNA molecules was achieved with this method.

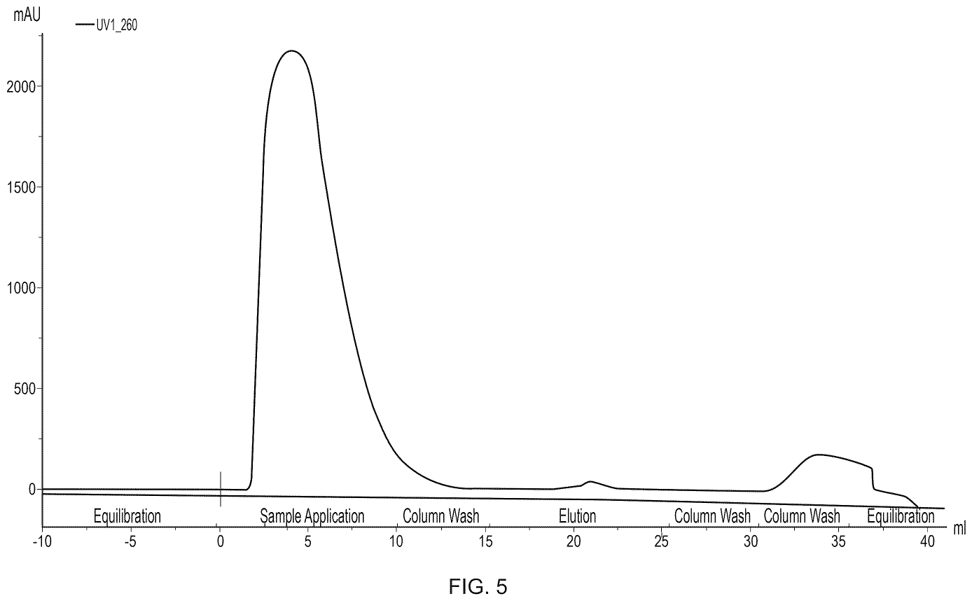

To determine the recovery of the DNA sample, following T5 exonuclease treatment and HIC, 2994 g of purified 2′-deoxyuridine (dU)-cheDNA molecules (comprising canonical uracil in one strand of the dsDNA) were injected into an HIC column. FIG. 5 shows the chromatogram of purified circular dU hemi-modified dsDNA molecules. Table 3 shows the amount injected and recovered from the column post-buffer exchanged, demonstrating that greater than 85% recovery of material post-chromatography and buffer exchange was achieved.

TABLE 3
Recovery of cheDNA molecules using HIC.

| | Concentration | | | Volume | Yield | Recovery |
|---|---|---|---|---|---|---|
| | (ng/μL) | A260/280 | A260/230 | (μL) | (μg) | (%) |
| cheDNA pre-HIC | 1555.2 | 2.02 | 2.28 | 1925 | 2994 | n/a |
| cheDNA post-HIC | 1610.3 | 2.02 | 2.29 | 1590 | 2560 | 85.52 |

### Example 3: Preparatory Scale Separation of Circular Double-Stranded DNA (cdsDNA) Molecules from Linear and Concatemer Species

This Example demonstrates enrichment of circular double-stranded DNA (cdsDNA) molecules from impurities, e.g., linear and concatemeric DNA molecules.

The synthesis of 1-5 kilobase (kb) cdsDNA molecules began with 12 to 16 hours of digestion at 37° C. with 80 rpm agitation of PCR product with a specific restriction enzyme (e.g., BsaI) corresponding to a restriction enzyme recognition sequence in the PCR product. The enzyme was then heat-inactivated, and the digested DNA molecule was circularized using T3 DNA ligase (M0317, New England Biolabs) in a bioreactor at a 80-rpm agitation at room temperature (23° C.+/−2° C.) to form cdsDNA molecules. T3 ligase was heat-inactivated at 65° C. for 3 hours. The ligated DNA molecule was concentrated, and the buffer was exchanged using a 10-50 kDa molecular weight cut-off (MWCO) hollow fiber with 7 diafiltration volumes of water.

The cdsDNA molecules were enriched from linear DNA molecules and linear concatemer byproduct impurities from the ligation reaction using T5 exonuclease treatment followed by enrichment using fast protein liquid chromatography (FPLC), specifically hydrophobic interaction chromatography (HIC). The linear DNA species were digested with 0.1-2.5 U/μg of T5 exonuclease enzyme at 37° C. and 80-rpm agitation with 1×NEB 4 Buffer and 2 mM Tris pH 10. The cdsDNA molecules were then purified using an AKTA Pure or AKTA Avant (Cytiva) with CIMmultus C4 HLD 1 mL hydrophobic interaction chromatography monolith columns (Bioseparation/Sartorius).

The DNA sample buffer was adjusted after T5 exonuclease treatment to match the equilibration/binding buffer for HIC (25 mM Tris, 1.25 M Ammonium sulfate, 10 mM EDTA, pH 7.0) and filtered through a 0.22 m filter. The column was equilibrated with 10 column volume (CV) of the equilibration/binding buffer at a 5 ml/CV flow rate. The T5 exonuclease-treated sample was loaded into the column via the sample pump, and the flow-through containing the circular DNA molecules was collected via Outlet 1. The flow-through material was concentrated in a Repligen system using a 10-30 kDa MWCO hollow fiber followed by 10 diafiltration volume into 1 mM sodium citrate pH 6.5. The purified circular dsDNA concentration was analyzed by nanodrop and high-performance liquid chromatography (HPLC). Over 90% purity of cdsDNA molecules was achieved with the described method. Over 76% recovery of material post-chromatography was achieved (Table 4).

TABLE 4
Recovery of cdsDNA molecules using HIC.

| | Concentration | | | Volume | Yield | Recovery |
|---|---|---|---|---|---|---|
| | (ng/μL) | A260/280 | A260/230 | (μL) | (μg) | (%) |
| DNA pre-HIC | 1207.80 | 1.89 | 2.14 | 4400 | 5314 | n/a |
| DNA post-HIC | 1469.25 | 1.91 | 2.30 | 2750 | 4040 | 76.03 |

### Example 4: Preparatory Scale Separation of Circular Hemi-Modified Double-Stranded DNA (cheDNA) from Linear and Concatemer Species

This Example demonstrates enrichment of circular hemi-modified double-stranded DNA (cheDNA) molecules from linear and concatemeric DNA molecules. The cheDNA molecules comprise chemically modified nucleotides (specifically, having chemically modified nucleobases) in one strand, e.g., sense strand, of the dsDNA molecule.

The 1-5 kb cheDNA molecules used in this experiment had an antisense strand that was free of chemically modified nucleobases and a sense strand comprising A, C, G, and either (i) 2′-deoxyuridine or (ii) 5-hydroxymethyl-2′-deoxyuridine. In other words, the cheDNA lacked thymine and instead comprised canonical uracil or 5-hydroxymethyluracil in the sense strand.

The cheDNA molecules were enriched from linear DNA and linear concatemer byproduct impurities from the ligation reaction using T5 exonuclease treatment followed by enrichment using FPLC, specifically HIC. The linear DNA species were digested with 0.1-2.5 U/μg of T5 exonuclease enzyme at 37° C. and 80 rpm agitation with 1×NEB 4 Buffer and 2 mM Tris pH 10. The cheDNA molecules were then purified using an AKTA Pure or AKTA Avant (Cytiva) with CIMmultus C4 HLD 1 mL hydrophobic interaction chromatography monolith columns (Bioseparation/Sartorius).

The DNA sample buffer was adjusted after T5 exonuclease treatment to match the equilibration/binding buffer for HIC (25 mM Tris, 1.25 M Ammonium sulfate, 10 mM EDTA, pH 7.0). The column was equilibrated with 10 column volumes (CV) of the equilibration/binding buffer at a 5 mL/CV flow rate. The T5 exonuclease-treated sample was filtered through a 0.22 μm filter and loaded into the column via the sample pump, and the flow-through containing the circular hemi-modified DNA molecules was collected via Outlet 1. The flow-through material was concentrated in a Repligen system using a 10-30 kDa MWCO hollow fiber followed by 7 diafiltration volumes into 1 mM sodium citrate pH 6.5. The purified circular hemi-modified dsDNA concentration was analyzed by nanodrop and HPLC. Over 90% purity of cheDNA molecules was achieved with this method. Over 99% recovery of material post-chromatography was achieved (Table 5).

TABLE 5
Recovery of cheDNA molecules using HIC.

| | Concentration | | | Volume | Yield | Recovery |
|---|---|---|---|---|---|---|
| | (ng/μL) | A260/280 | A260/230 | (μL) | (μg) | (%) |
| cheDNA pre-HIC | 1048.75 | 2.01 | 2.36 | 17000 | 17829 | n/a |
| cheDNA post-HIC | 1006.80 | 1.99 | 2.32 | 17615 | 17735 | 99.47 |

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

1. A method of making a composition comprising purified DNA molecules, the method comprising:
a) providing a solution comprising a plurality of circular, double-stranded DNA (cdsDNA) molecules, wherein the cdsDNA molecules lack one or both of a bacterial origin of replication and a selectable marker;
b) contacting the solution to a hydrophobic interaction chromatography (HIC) matrix; and
c) collecting at least some of the cdsDNA molecules from the matrix;
thereby making the composition comprising purified DNA molecules.

2. A method of making a composition comprising purified DNA molecules, the method comprising:
a) providing a solution comprising a plurality of circular, double-stranded DNA (cdsDNA) molecules;
b) subjecting the solution to conditions having exonuclease activity;
c) contacting the solution to a hydrophobic interaction chromatography (HIC) matrix; and
d) collecting at least some of the cdsDNA molecules from the matrix;
thereby making the composition comprising purified DNA molecules.

3. The method of claim 1, wherein:
(i) the cdsDNA molecules are not supercoiled; or
(ii) the cdsDNA molecules substantially lack a material portion of a vector backbone.

4. (canceled)

5. The method of claim 1, wherein:
(i) at least 85%, at least 90%, at least 95%, at least 96%, at least 97%, at least 98%, at least 99%, at least 99.5%, or at least 99.6% by mass of total DNA molecules in the composition are the cdsDNA molecules,
(ii) 85%-90%, 90%-95%, 95%-96%, 96%-97%, 97%-98%, 98%-99%, or 99%-99.6% by mass of total DNA molecules in the composition are the cdsDNA molecules;
(iii) the composition comprises between 1 μg and 500 μg, between 500 μg and 1 mg, between 1 mg and 5 mg, between 5 mg and 10 mg, between 10 mg and 15 mg, or between 10 mg and 20 mg of the cdsDNA molecules; or
(iv) the composition comprises less than:
15%, 10%, 5%, 4%, 3%, 2%, 1%, or 0.5% of linear DNA molecules by mass;
15%, 10%, 5%, 4%, 3%, 2%, 1%, or 0.5% of linear concatemeric DNA molecules by mass; and/or
15%, 10%, 5%, 4%, 3%, 2%, 1%, or 0.5% of circular concatemeric DNA molecules by mass.

6.-7. (canceled)

8. The method of claim 1, wherein the mass of cdsDNA molecules in the composition comprising purified DNA molecules is 50%-60%, 60%-65%, 65%-70%, 70%-75%, 75%-80%, 80%-85%, 85%-90%, 90%-95%, 95%-99%, or 99%-99.5% of the mass of cdsDNA molecules in the solution prior to contacting the solution to the HIC matrix.

9. The method of claim 1, wherein the solution of a) was not produced in a microorganism.

10. The method of claim 1, wherein prior to contacting the solution to the HIC matrix:
(i) the solution comprises 20-50 mM Tris, 1.0-2.0 M ammonium sulfate, and 5-15 mM EDTA, and wherein the solution has a pH of 7.0-8.0;
(ii) the solution was contacted with a filter; or
(iii) the HIC matrix was contacted with an equilibration buffer comprising 20-50 mM Tris, 1.0-2.0 M ammonium sulfate, and 5-15 mM EDTA, and wherein the equilibration buffer comprises a pH of 7.0-8.0.

11.-12. (canceled)

13. The method of claim 1, wherein collecting at least some of the cdsDNA molecules from the matrix comprises collecting a flow-through from the matrix, wherein the flow-through comprises the cdsDNA molecules.

14. (canceled)

15. The method of claim 1, wherein, following collecting at least some of the cdsDNA molecules from the matrix;
(i) the cdsDNA molecules are filtered and/or concentrated; or
(ii) the cdsDNA molecules are contacted with a solution comprising sodium citrate.

16. The method of claim 1, wherein:
(i) the HIC matrix comprises a butyl (C4) ligand; or
(ii) the HIC matrix is comprised in a column.

17. (canceled)

18. The method claim 1, further comprising measuring one or more of the following in the composition comprising purified DNA molecules:
(a) the mass % of cdsDNA molecules;
(b) the mass % of linear DNA molecules;
(c) the mass % of linear concatemeric DNA molecules;
(d) the mass % of circular concatemeric DNA molecules;
(e) the length of cdsDNA molecules;
(f) endotoxin levels; or
(g) amount of protein levels or activity.

19. The method of claim 18, further comprising formulating the composition as a drug product if the one or more measured parameters (a)-(g) meet a predetermined value.

20. A composition or a pharmaceutical composition comprising the purified DNA molecules produced by the method of claim 1.

21. A composition or a pharmaceutical composition comprising circular, double-stranded DNA (cdsDNA) molecules, wherein:
i) the cdsDNA molecules lack one or both of a bacterial origin of replication and a selectable marker; and
ii) at least 85%, at least 90%, at least 95%, at least 96%, at least 97%, at least 98%, at least 99%, or at least 99.5% by mass of total DNA molecules in the composition are the cdsDNA molecules.

22. The method of claim 1, wherein the cdsDNA molecules:
(i) comprise a chemically modified nucleotide; or
(ii) have a length of at least 500, at least 1000, at least 2000, at least 3000, at least 4000, at least 5000, at least 6000, at least 7000, at least 8000, at least 9000, or at least 10000 nucleotides.

23. The method of claim 1, wherein the cdsDNA molecules comprise a sense strand and an antisense strand, wherein the sense strand comprises a chemically modified nucleotide, and the antisense strand is substantially free of chemically modified nucleotides.

24. The method of claim 23, wherein 1-25%, 25-50%, 50-75%, or 75-100% of nucleotides in the sense strand are chemically modified nucleotides.

25. (canceled)

26. The method of claim 1, wherein the cdsDNA molecules comprise a promoter sequence and an effector sequence that encodes an effector.

27. The method of claim 26, wherein the effector is a polypeptide or an RNA.

28. (canceled)

29. A method of expressing an effector in a target cell, delivering an effector to a target cell, modulating a biological activity in a target cell, or treating a cell, tissue, or subject in need thereof, the method comprising:
introducing into a target cell, or administering to the cell, tissue, or subject, the composition of claim 20, wherein the cdsDNA molecules comprise an effector sequence encoding an effector;
thereby expressing the effector in the target cell, delivering the effector to the target cell, modulating the biological activity in the target cell, or treating the cell, tissue, or subject.

30.-32. (canceled)

33. A composition or a pharmaceutical composition comprising the purified DNA molecules produced by the method of claim 2.

34. A method of expressing an effector in a target cell, delivering an effector to a target cell, modulating a biological activity in a target cell, or treating a cell, tissue, or subject in need thereof, the method comprising:
introducing into a target cell, or administering to the cell, tissue, or subject, the composition of claim 33, wherein the cdsDNA molecules comprise an effector sequence encoding an effector;
thereby expressing the effector in the target cell, delivering the effector to the target cell, modulating the biological activity in the target cell, or treating the cell, tissue, or subject.

35. A method of expressing an effector in a target cell, delivering an effector to a target cell, modulating a biological activity in a target cell, or treating a cell, tissue, or subject in need thereof, the method comprising:
introducing into a target cell, or administering to the cell, tissue, or subject, the composition of claim 21, wherein the cdsDNA molecules comprise an effector sequence encoding an effector;
thereby expressing the effector in the target cell, delivering the effector to the target cell, modulating the biological activity in the target cell, or treating the cell, tissue, or subject.
